(19) United States
(12) Patent Application Publication
(10) Pub. No.: US 2023/0126784 A1
(43) Pub. Date: Apr. 27, 2023

(54) A METHOD TO GENERATE CHIMERIC ANTIGEN RECEPTOR (CAR) T-CELLS (CAR-T CELLS) FROM PATHOGEN-SPECIFIC CYTOTOXIC LYMPHOCYTES TO ENABLE THE SUBSEQUENT IN VIVO MODULATION OF THEIR FUNCTIONAL ACTIVITY

(71) Applicant: ALBERT EINSTEIN COLLEGE OF MEDICINE, Bronx, NY (US)

(72) Inventors: Harris GOLDSTEIN, Bronx, NY (US); Steven C ALMO, Bronx, NY (US); Scott GARFORTH, Bronx, NY (US); Mengyan LI, Bronx, NY (US)

(21) Appl. No.: 17/905,325

(22) PCT Filed: Mar. 8, 2021
(86) PCT No.: PCT/US2021/021411

§ 371 Date: Aug. 30, 2022

Related U.S. Application Data
(60) Provisional application No. 62/986,572, filed on Mar. 6, 2020.

(51) Int. Cl. A61K 35/17; C12N 15/86; C07K 14/74; C07K 14/725; C07K 14/005; C07K 16/28; A61P 35/00; A61P 35/02; C12N 5/0783

(52) U.S. Cl. CPC A61K 35/17; C12N 15/86; C07K 14/70539; C07K 14/7051; C07K 14/005; C07K 16/2818; C07K 16/2878; A61P 35/00; A61P 35/02; C12N 5/0636; C12N 2710/16122; C12N 2710/16134; C12N 2740/15043; C07K 2319/00; A61K 38/00

(57) ABSTRACT

The present disclosure relates to a method of modulating one or more genetically modified cells, e.g., chimeric antigen receptor (CAR)-expressing cells, ex vivo and/or in vivo.

### CROSS-REFERENCE TO RELATED APPLICATION

This application claims priority to U.S. Provisional Application No. 62/986,572 filed on Mar. 6, 2020, the disclosure of which is hereby incorporated by reference herein in its entirety.

### STATEMENT REGARDING FEDERALLY SPONSORED RESEARCH

This invention was made with government support under R01CA198095-02 and 1R01AI145024-01 awarded by the National Institute of Health/National Cancer Institute (NIH/NCI). The government has certain rights in the invention.

### SEQUENCE LISTING

This disclosure includes a sequence listing, which was submitted in ASCII format via EFS-Web, and is hereby incorporated by reference in its entirety. The ASCII copy, created on Mar. 8, 2021, is named Sequence_Listing_8003WO00. and is 1.74 kb in size.

### BACKGROUND

Chimeric antigen receptor (CAR) modified T-cells (CAR-T cells) or modified natural killer (NK) cells are a therapeutic approach that enables the mobilization of the immune response to treat previously refractory B-cell malignancies. In CAR-T cells, instead of the standard T cell receptor (TCR) MHC-restricted antigen recognition molecule expressed by T cells, CAR-T cells express an MHC-independent recombinant CAR consisting of an extracellular antibody-derived or ligand-derived binding fragment and an intracellular domain which can include TCR zeta chain activation domains and costimulatory signaling domains such as CD28 and/or 4-1BBL. For example, if the CAR is an antibody-derived binding fragment which binds a target antigen such as CD19, a molecule expressed by B cells, recognition by the CAR of the CD19-expressing B cell will result in activation of the CAR-T cell via signal transduction triggered by the intracellular signaling domains. After recognition of CD19 expressed by the B cells, these CD19-specific CAR-T cells will eliminate the targeted B cells, including malignant B cells which cause leukemia and lymphoma. This approach has revolutionized therapy for pre-B cell acute lymphoblastic leukemia (ALL) and B cell lymphomas by providing a new strategy to eliminate malignant B cells and cure patients with ALL or B cell lymphoma refractory to previous treatments. CAR-expressing cells can also be engineered to treat infectious diseases. For example, CARs can be engineered to recognize HIV antigens expressed on the surface of infected cells, in an MHC-independent manner, and stimulate the CAR-T cell through intracellular signaling domains that include TCR activation and costimulatory domains.

First-generation HIV-specific CAR-T cells transferred into HIV-infected individuals displayed long-term persistence but no significant clinical effect. Subsequently, the ex vivo activity of HIV-specific CAR-T cells was markedly improved by using enhanced immunoreceptors and by enhancing the resistance of the CAR-T cells to HIV infection by ablating expression of CCR5, the primary HIV coreceptor. In addition to its established efficacy for the treatment of malignancies, these improvements have also made CAR-T cells an attractive therapeutic modality to contribute to the functional cure of HIV infection. However, analyses of long-term outcomes of CAR-T cell treatments for cancer have revealed that the disease remission induced by CAR-T cell therapy frequently does not persist due to poor maintenance of CAR-T cell function or the emergence of resistant cancer cells. Furthermore, off-target activity or overly enhanced immune activation by CAR-T cells may cause toxicity requiring supportive therapies and would benefit from the rapid inhibition of CAR-T cell activity. However, at present, once CAR-T cells are infused into patients, there is no available strategy to specifically amplify or inhibit their activities. In addition, the costimulatory signals required for T cell activation, expansion, and function in current CAR-T cells are provided by integrating the costimulatory signaling domains into the CAR construct, which limits modulation of CAR-T cell function to the small number (e.g., 1 or 2) costimulatory domains directly integrated into the CAR construct. Developing a method to specifically deliver additional endogenous modulatory signals (i.e., not encoded within the CAR) to CAR-T cells after infusion would provide the full spectrum of T cell functionality to enable the in vivo reactivation and amplification of their capacity to eliminate target cells, or to suppress their activities if the CAR-T cells are mediating toxic effects. These endogenous functions include, but are not limited to, costimulatory signals (e.g., ICOS, CD40L, OX40, CD27 and GITR), coinhibitory signals (e.g., PD-1, CTLA-4 and TIM3) and/or a variety of cytokine signals (e.g., IL-2, IL-7, IL-12 and IL-15).

Accordingly, there exists a need to modulate function of CAR-expressing cells (e.g., CAR-T cells) following transfer to a patient, either to boost its activity by reactivating the CAR-expressing cells or suppressing activity if the CAR-expressing cells are mediating toxic effects (e.g., cytokine storms or off-target cell killing).

### SUMMARY

In some embodiments, the disclosure relates to a method of modulating one or more genetically modified cells ex vivo and/or in vivo, the method comprising: contacting a cell expressing a receptor that recognizes a cognate peptide with a fusion protein, the fusion protein having at least one major histocompatibility complex (MHC) molecule and at least one cognate peptide, the cognate peptide capable of being recognized by the receptor; culturing the cell for a period of time to generate a plurality of cells, the plurality of cells having a receptor that recognizes the cognate peptide; transducing the plurality of cells with a polynucleotide encoding a chimeric antigen receptor (CAR) to generate a plurality of CAR-expressing cells having both the receptor that recognizes the cognate peptide and the CAR; and contacting the plurality of CAR-expressing cells with the fusion protein to modulate the CAR-expressing cells. In some embodiments, the cognate peptide is 6 to 18 amino acid residues in length, i.e., 6, 7, 8, 9, 10, 11, 12, 13, 14, 15, 16, 17, or 18 amino acid residues. In some embodiments, the cognate peptide includes but is not limited to HIV Gag 77-85 (SL9) (SLYNTVATL; SEQ ID NO: 1) or SL9 variant (SLFNTIAVL; SEQ ID NO: 2), HIV Nef 190-198 (AFHHVAREL) (SEQ ID NO: 3), HIV reverse transcriptase 476-484 (ILKEPVHGV; SEQ ID NO: 4), HIV reverse transcriptase 179-187 (VIYQMDDL; SEQ ID NO: 5), CMV pp65 (495-503) (NLVPMVATV; SEQ ID NO: 6), and EBV LMP-2 (426-434) (CLGGLLTMV; SEQ ID NO: 7), or a peptide having an amino acid sequence at least 75%, at least 80%, at least 85%, at least 90%, or at least 95% identical to the sequence of one of the aforementioned peptides.

In some embodiments, this disclosure relates to modulated CAR-expressing cells produced by the methods disclosed herein.

In certain embodiments, the cell is selected from a T cell, a natural killer (NK) cell, a memory CD8 cell, or a peripheral blood mononuclear cell (PBMC).

In certain embodiments, the fusion protein is a synTac.

In certain embodiments, the plurality of CAR-expressing cells is contacted with the fusion protein which elicits an in vivo or ex vivo expansion or modulation of the plurality of CAR-expressing cells.

In some embodiments, modulating the CAR-expressing cell induces proliferation of the CAR-expressing cell, induces differentiation of the CAR-expressing cell, stimulates the CAR-expressing cell to induce cytotoxic effects, inhibits the CAR-expressing cell, induces apoptosis or necrosis in the CAR-expressing cell, induces quiescence, or any combination thereof.

In certain embodiments, the CAR-expressing cell is selected from one of the following: a T cell, an NK cell, or a PBMC.

In certain embodiments, the CAR-expressing cell is a T cell, wherein the T cell is selected from one of the following: a CD4⁺ T cell, a CD8⁺ T cell, a CD4″ CD8″ double negative T cell.

In certain embodiments, the CAR-expressing cell is an NK cell, wherein the NK cell is selected from one of the following: a CD3⁻ NK cell, a CD56/NCAM1⁺ NK cell, a CD94⁺ NK cell, a CD122/IL-2 R beta⁺ NK cell, a CD127/IL-7 R alpha-NK cell, an Fc gamma RIII/CD16^{+/-} NK cell, a KIR family receptor⁺ NK cell, an NKG2A⁺ NK cell, an NKG2D⁺ NK cell, an NKp30⁺ NK cell, an NKp44⁺ NK cell, an NKp46⁺ NK cell, an NKp80⁺ NK cell, a CD11b/Integrin alpha M⁺ NK cell, a CD27⁺ NK cell, a CD161/NK1.1⁺ NK cell, an Integrin alpha 2/CD49b⁺ NK cell, or a Ly49 family receptor⁺ NK cell.

In certain embodiments, the CAR-expressing cell is stimulated to induce cytotoxic effects in a target cell. In certain embodiments, the CAR-expressing cell secretes a molecule that may be selected from the following: a granzyme, a perforin, a cytokine, a Fas Ligand (FasL).

In certain embodiments, the fusion protein is specific for a eukaryotic pathogen, wherein the cognate peptide of the fusion protein is encoded by a sequence derived from the eukaryotic pathogen. In certain aspects, the eukaryotic pathogen is selected from a virus, a fungus, or a protozoa.

In certain embodiments, the fusion protein is a virus-specific fusion protein, wherein the cognate peptide of the fusion protein is encoded by a sequence derived from a virus. In certain aspects, the virus is selected from the group consisting of a human immunodeficiency virus (HIV), a cytomegalovirus (CMV), an Epstein-Barr virus (EBV), a measles virus, a mumps virus, a varicella virus, an influenza virus, a rotavirus, a polio virus, a hepatitis virus, a diphtheria virus, a pertussis virus, a pneumococcal virus, a meningococcal fusion protein, a zoster virus, a yellow fever virus, a human papillomavirus, a rabies virus, or the like. In some embodiments, the cognate peptide is 6 to 18 amino acid residues in length, i.e., 6, 7, 8, 9, 10, 11, 12, 13, 14, 15, 16, 17, or 18 amino acid residues. In some embodiments, the cognate peptide includes but is not limited to HIV Gag 77-85 (SL9) (SLYNTVATL; SEQ ID NO: 1) or SL9 variant (SLFNTIAVL; SEQ ID NO: 2), HIV Nef 190-198 (AFHHVAREL) (SEQ ID NO: 3), HIV reverse transcriptase 476-484 (ILKEPVHGV; SEQ ID NO: 4), HIV reverse transcriptase 179-187 (VIYQMDDL; SEQ ID NO: 5), CMV pp65 (495-503) (NLVPMVATV; SEQ ID NO: 6), and EBV LMP-2 (426-434) (CLGGLLTMV; SEQ ID NO: 7), or a peptide having an amino acid sequence at least 75%, at least 80%, at least 85%, at least 90%, or at least 95% identical to the sequence of one of the aforementioned peptides.

In certain embodiments, the fusion protein is a fungus-specific fusion protein, wherein the cognate peptide of the fusion protein is encoded by a sequence derived from a fungus. In certain aspects, the fungus is selected from the group consisting of Basidiomycota, Sac fungi, Eomycota, Zygomycota, Chytridiomycota, Microsporidia, Glomeromycota, Hyphomycetes, Neocallimastigomycota, Blastocladiomycota, Kickxellomycotina, Inocybe salicis, Babjeviella inositovra, Aspergillus, Blastomyces, Cryptococccus, Candida, Coccidioides, Histoplasma, Sporothrix, or the like.

In certain embodiments, the fusion protein is a protozoan-specific fusion protein, wherein the cognate peptide of the fusion protein is encoded by a sequence derived from a protozoa. In certain aspects, the protozoa is selected from the group consisting of Leishmania, Cryptosporidium, Balantidium, Toxoplasma gondii, Plasmodium, Balantidium coli, Entamoeba, Entamoeba coli, Entamoeba histolytica, Entamoeba gingivalis, Entamoeba invadens, Entamoeba poleck, Giardia lamblia, Acanthamoeba, Endolimax, Endolimax nana, Iodamoeba, Iodamoeba bütschlii, Archamoebae, Entamoeba moshkovskii, Tritrichomonas, Tritrichomonas foetus, Trichomonas vaginalis, Enteromonas hominis, Entromonas, Entamoebidae, Hartmannella, Entomophthorales, Conidiobolus, Basidiobolus ranarum, Hexamitidae, Amoebida, Retortamonad, Hartminnellidae, Acanthamoebidae, Paramoeba, Conidiobolus incongruus, Conidiobolus coronatus, Malpighamoeba, or the like.

In some embodiments, the fusion protein is a bacteria-specific fusion protein, wherein the cognate peptide of the fusion protein is encoded by a sequence derived from a bacterium. In certain aspects, the bacterium is selected from the group consisting of Escherichia, Escherichia coli, MRSA, Shigella, Salmonella, Pseudomonas, Pseudomonas aeruginosa, Streptococcus, Streptococcus pneumoniae, Asiatic cholera, Listeria monocytogenes, Mycobacterium tuberculosis, Yersinia, Campylobacter, Klebsiella pneumoniae, Entrococcus, Neisseria, Enterobacteriaceae, Group A streptococcus, Legionella pneumophila, Corynebacterium, Staphylococcus aureus, Mycobacterium, Shigella, Helicobacter, O157:H7, Anthrax bacterium, Clostridium botulinum, Salmonella enterica, Yersinia pestis, Clostridium perfringens, Haemophilus influenzae, Rickettsia, Acinetobacter, Campylobacter jejuni, Burkholderia, Haemophilus, Enterococus faecalis, Chlamydia, Bordatella pertussis, Clostridioides difficile, Enterococcus faecium, Klebs-Löffler bacillus, Clostridium tetani, Treponema, Acinetobacter baumanni, Brucella, Treponemia pallidum, Xanthomonas, or the like.

In some embodiments, the method further comprises harvesting the cell from a subject.

In some embodiments, the method further comprises vaccinating the subject with a vaccine containing the cognate peptide recognized by the receptor prior to harvesting the cell. In some embodiments, the cognate peptide is 6 to 18 amino acid residues in length, i.e., 6, 7, 8, 9, 10, 11, 12, 13, 14, 15, 16, 17, or 18 amino acid residues. In some embodiments, the cognate peptide includes but is not limited to HIV Gag 77-85 (SL9) (SLYNTVATL; SEQ ID NO: 1) or SL9 variant (SLFNTIAVL; SEQ ID NO: 2), HIV Nef 190-198 (AFHHVAREL) (SEQ ID NO: 3), HIV reverse transcriptase 476-484 (ILKEPVHGV; SEQ ID NO: 4), HIV reverse transcriptase 179-187 (VIYQMDDL; SEQ ID NO: 5), CMV pp65 (495-503) (NLVPMVATV; SEQ ID NO: 6), and EBV LMP-2 (426-434) (CLGGLLTMV; SEQ ID NO: 7), or a peptide having an amino acid sequence at least 75%, at least 80%, at least 85%, at least 90%, or at least 95% identical to the sequence of one of the aforementioned peptides.

In certain embodiments, the method further comprises purifying the plurality of CAR-expressing cells.

In certain embodiments, the method further comprising administering at least one of the plurality of CAR-expressing cells to a subject. In certain aspects, the CAR-expressing cell is an autologous cell or an allogeneic cell. In some embodiments, the method further comprises administering to the subject the fusion protein after infusion of the CAR-expressing cell to elicit in vivo modulation of the CAR-expressing cell.

In certain embodiments, the in vivo modulation of the CAR-expressing cell induces proliferation of the CAR-expressing cell, induces differentiation of the CAR-expressing cell, stimulates the CAR-expressing cell to induce cytotoxic effects, inhibits the CAR-expressing cell, induces apoptosis or necrosis in the CAR-expressing cell, induces quiescence, or any combination thereof.

In some embodiments, the fusion protein further comprises an additional moiety, wherein the additional moiety comprises a modulatory domain, a co-stimulatory ligand, a co-stimulatory receptor recruitment molecule, a cytokine, a modulatory cytokine, a cytokine receptor engager, a co-inhibitory ligand, a co-inhibitory receptor recruitment molecule, an affinity reagent, or any combination thereof. In certain embodiments, the co-stimulatory or co-inhibitory receptor recruitment molecule may be a single chain Fv (scFv) molecule. In some aspects, the scFv molecule may be capable of binding a co-stimulatory receptor recruitment molecule or co-inhibitory receptor recruitment molecule selected from the group consisting of an scFv molecule recognizing CD28, 4-1BB, GITR, CD27, ICOS, CD40L, TIM3, OX-40, PD-L1, CTLA4, B7-H1, FasL, PD-1, or the like. In certain embodiments, the co-stimulatory receptor recruitment molecule or co-inhibitory recruitment molecule may be a ligand or an affinity molecule. In some aspects, the ligand or affinity molecule is selected from the group consisting B7-1, B7-2, B7-H2, CD40, B7-H1, PD1-1, PDL-2, 4-1BBL, GITRL, CD70, ICOS-L, OX-40L, Fas, or the like. In certain aspects, the cytokine is selected from the group consisting IL-2, IL-4, IL-6, IL-7, IL-10, IL-12, IL-15, IL-17, IL-21, IL-23, IFN-gamma, TGF-beta, or the like.

In some embodiments, the modulation of the CAR-expressing cell may be via direct binding of the CAR-expressing cell.

In some embodiments, the subject has a disease associated with expression of a tumor antigen, a disease associated with a pathogen, or an autoimmune disease or disorder. In certain embodiments, the disease associated with expression of a tumor antigen is a proliferative disease, a precancerous condition, a cancer, or a non-cancer related indication associated with expression of the tumor antigen.

In certain embodiments, the subject has a cancer selected from the group consisting of chronic lymphocytic leukemia (CLL), acute leukemia, acute lymphoid leukemia (ALL), B-cell acute lymphoid leukemia (B-ALL), T-cell acute lymphoid leukemia (T-ALL), chronic myelogenous leukemia (CIVIL), B cell prolymphocytic leukemia, blastic plasmacytoid dendritic cell neoplasm, Burkitt's lymphoma, diffuse large B cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia. In certain embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of the cancers, and metastatic lesions of the cancers. In certain embodiments, the cancer is leukemia or lymphoma. In certain embodiments, wherein the lymphoma is lymphoblastic lymphoma or B-cell Non-Hodgkin's lymphoma.

In some embodiments, the subject may have a disease associated with a pathogen, wherein the pathogen is a eukaryotic pathogen. In some aspects, the eukaryotic pathogen is selected from a virus, a fungus, or a protozoa. In certain aspects, the eukaryotic pathogen is a virus. In certain aspects, the virus is selected from the group consisting of a human immunodeficiency virus (HIV), a cytomegalovirus (CMV) (e.g., CMV-pp65), an Epstein-Barr virus (EBV), a measles virus, a mumps virus, a varicella virus, an influenza virus, a rotavirus, a polio virus, a hepatitis virus, a diphtheria virus, a pertussis virus, a pneumococcal virus, a meningococcal fusion protein, a zoster virus, a yellow fever virus, a human papillomavirus, a rabies virus, or the like. In certain aspects, the eukaryotic pathogen is a fungus. In certain aspects, the fungus is selected from the group consisting of Basidiomycota, Sac fungi, Eomycota, Zygomycota, Chytridiomycota, Microsporidia, Glomeromycota, Hyphomycetes, Neocallimastigomycota, Blastocladiomycota, Kickxellomycotina, Inocybe salicis, Babjeviella inositovra, or the like. In certain aspects, the eukaryotic pathogen is a protozoa. In certain aspects, the protozoa is selected from the group consisting of Toxoplasma gondii, Plasmodium, Balantidium coli, Entamoeba, Entamoeba coli, Entamoeba histolytica, Entamoeba gingivalis, Entamoeba invadens, Entamoeba poleck, Giardia lamblia, Acanthamoeba, Endolimax, Endolimax nana, Iodamoeba, Iodamoeba butschlii, Archamoebae, Entamoeba moshkovskii, Tritrichomonas, Tritrichomonas foetus, Trichomonas vaginalis, Enteromonas hominis, Entromonas, Entamoebidae, Hartmannella, Entomophthorales, Conidiobolus, Basidiobolus ranarum, Hexamitidae, Amoebida, Retortamonad, Hartminnellidae, Acanthamoebidae, Paramoeba, Conidiobolus incongruus, Conidiobolus coronatus, Malpighamoeba, or the like. In certain aspects, the pathogen is a bacterial pathogen. In certain aspects, the bacterial pathogen is selected from the group consisting of Escherichia, Escherichia coli, MRSA, Shigella, Salmonella, Pseudomonas, Pseudomonas aeruginosa, Streptococcus, Streptococcus pneumoniae, Asiatic cholera, Listeria monocytogenes, Mycobacterium tuberculosis, Yersinia, Campylobacter, Klebsiella pneumoniae, Entrococcus, Neisseria, Enterobacteriaceae, Group A streptococcus, Legionella pneumophila, Corynebacterium, Staphylococcus aureus, Mycobacterium, Shigella, Helicobacter, O157:H7, Anthrax bacterium, Clostridium botulinum, Salmonella enterica, Yersinia pestis, Clostridium perfringens, Haemophilus influenzae, Rickettsia, Acinetobacter, Campylobacter jejuni, Burkholderia, Haemophilus, Enterococus faecalis, Chlamydia, Bordatella pertussis, Clostridioides difficile, Enterococcus faecium, Klebs-Löffler bacillus, Clostridium tetani, Treponema, Acinetobacter baumanni, Brucella, Treponemia pallidum, Xanthomonas, or the like.

In some embodiments, the subject experiences a reduction or elimination in an inflammatory response, a cytokine storm, or at least one off-target effect, or any combination thereof as compared to a control subject.

In some embodiments, the subject experiences a reduction in a symptom or a side effect as compared to a control subject. In certain aspects, the symptom is cytokine-release syndrome (CRS), a neurologic toxicity, B-cell aplasia, tumor lysis syndrome (TLS), anaphylaxis, fever, joint/muscle aches, shortness of breath, low blood pressure, confusion, or a seizure.

This listing is intended to be exemplary and illustrative rather than comprehensive and limiting. Additional aspects and embodiments may be set out in, or apparent from, the remainder of this disclosure and the claims.

### BRIEF DESCRIPTION OF THE DRAWINGS

The accompanying drawings exemplify certain aspects and embodiments of the present disclosure. The depictions in the drawings are intended to provide illustrative, and schematic rather than comprehensive, examples of certain aspects and embodiments of the present disclosure. The drawings are not intended to be limiting or binding to any particular theory or model and are not necessarily to scale. Without limiting the foregoing, nucleic acids and polypeptides may be depicted as linear sequences, or as schematic, two- or three-dimensional structures; these depictions are intended to be illustrative, rather than limiting or binding to any particular model or theory regarding their structure.

FIG. 1 depicts a schematic overview of a method according to one embodiment of the present disclosure. The schematic overview depicts the following steps: (1) T cells isolated from the patient are (2) treated with a p65-specific modulatory fusion protein to expand the population of pp65-specific CD8 T cells and then (3) transduced with a lentivirus expressing CAR followed by (4) further ex vivo expansion and then (5) infusion into the patient, (6) Weeks to months later, these pp65/CAR-T cell can be reactivated and expanded by in vivo treatment with pp65-specific fusion protein.

FIGS. 2A and 2B depict a structural representation of a pp65-specific α-CD28 fusion modulatory protein and a α-CD19 CAR protein. FIG. 2A is a schematic showing the pp65-specific α-CD28 fusion modulatory protein constructed as a split sc-pMHC-Fc fusion protein, with the β2M, the MHC HLA-A*0201 alpha chain and SL9 peptide linked through engineered interchain disulfide bonds, and the αCD28 modulatory domains linked to the carboxy end of the β2M. FIG. 2B is a schematic showing a third-generation α-CD19-CAR or an α-CD19-CAR with an integrated myc tag.

FIG. 3 depicts a series of plots showing the expression of the GFP reporter gene correlates with expression of the α-CD19-CAR. T cells were transduced with lentivirus expressing either a α-CD19-CAR or an α-CD19-CAR with an integrated myc tag, both of which also have a GFP reporter gene. Four days after transduction the CD8 T cells were gated and analyzed by flow cytometry for transduction based on their expression of GFP and expression of the extracellular αCD19 using a CD19 detection reagent (Miltenyi Biotec, Cambridge, Mass.).

FIGS. 4A and 4B depict experimental results showing selective expression of CAR-CD19 by pp65-specific CD8 T cells. Peripheral blood mononuclear cells were treated with either α-CD3/α-CD28 for 3 days (FIG. 4A) or a pp65-αCD28 fusion protein (0.1 nM) for 7 days (FIG. 4B). The activated cells were either untransduced or transduced with a lentivirus expressing CD19-CAR with a GFP reporter or CD19-CAR with a GFP and myc tag reporter. Four days later, the cells were analyzed by flow cytometry for expression of CD8 and then the CD8+ population was gated and analyzed for pp65 tetramer binding and GFP expression.

FIGS. 5A and 5B depict experimental results showing selective expression of CAR-CD19 by pp65-specific CD8 T cells. Peripheral blood mononuclear cells were treated with either α-CD3/α-CD28 for 3 days (FIG. 5A) or a pp65-αCD28 fusion protein (0.1 nM) for 12 days (FIG. 5B). The activated cells were either untransduced or transduced with a lentivirus expressing CD19-CAR with a GFP reporter or CD19-CAR with a GFP and myc tag reporter. Four days later the cells were analyzed by flow cytometry for expression of CD8 and then the CD8+ population was gated and the fraction of cells binding the pp65 tetramer binding and expressing GFP are shown.

FIG. 6 shows that pp65-αCD28-synTac treatment selectively activated CMV-specific CD8+ T cells to enable their specific conversion into CD19 CAR T-cells. Representative dot plots showing the expansion of CMV-specific CD19 CAR-T cells by pp65-αCD28 synTac-stimulation of purified CD8+ T cells from an HLA-A*0201 donor who also had a defined population of CMV-pp65-specific CD8+ T cells and selective transduction with the CAR lentivirus of pp65-specific CD8+ T cells after treatment with pp65-αCD28 synTac. Transduction efficiency was indicated by degree of GFP expression (X axis), as the lentiviral vector used to transduce these PBMCs expressed a GFP reporter gene. Specificity for pp65 was determined by staining with a p65-specific tetramer (Y axis). Transduction efficiency was measured 3 days after transduction and 10 days after synTac stimulation or αCD3/αCD28-conjugated T Cell TransAct nanomatrix stimulation.

FIG. 7 shows that pp65-αCD28 synTac expanded CD19 CAR T-cells from an HLA-A*0201 donor more potently killed purified CD19 cells in vitro than CAR T-cells activated by standard αCD3/αCD28. CD8+ T cells were activated with either standard αCD3/αCD28 or stimulated with pp65-αCD28 synTac, followed by transduction with a lentivirus expressing a CD19 CAR. Five days after transduction, CD19 CAR T-cells were co-cultured for 24 hours with purified CD19 cells that were labeled with a cell membrane label (PKH26) at the indicated E/T ratios. Histograms shown were gated from CD19+ population and data shown represent percentage of dead CD19 cells for each condition and E/T ratio.

FIG. 8 shows that pp65-αCD28 synTac expanded CD19 CAR T-cells displayed greater production of INF-γ after incubation with B cells. CD8+ T cells were activated with either standard αCD3/αCD28 or stimulated with pp65-αCD28 synTac, followed by transduction with a lentivirus expressing a CD19 CAR and GFP reporter gene. Five days after transduction, CD19 CAR T-cells were co-cultured for 24 hours with purified CD19 cells at the E/T ratios indicated, or with PMA/Ionomycin as a positive control. Dot plots shown were gated from GFP+/pp65-tetramer+ population for the transduced conditions and from pp65-tetramer+ population for the untransduced conditions. Intracellular cytokine staining of IFN-γ and TNF-α is shown.

### DETAILED DESCRIPTION

### Definitions and Abbreviations

Unless otherwise specified, each of the following terms has the meaning set forth in this section.

The indefinite articles “a” and “an” denote at least one of the associated noun and are used interchangeably with the terms “at least one” and “one or more.” For example, the phrase “a module” means at least one module, or one or more modules.

The conjunctions “or” and “and/or” are used interchangeably.

“Domain” is used to describe a segment of a protein or nucleic acid. Unless otherwise indicated, a domain is not required to have any specific functional property.

“Subject” means a human, mouse, or non-human primate. A human subject can be any age (e.g., an infant, child, young adult, or adult), and may suffer from a disease, such as a cancer.

“Treat,” “treating,” and “treatment” as used herein mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” as used herein means the prevention of a disease in a subject, e.g., in a human, including (a) avoiding or precluding the disease; (b) affecting the predisposition toward the disease; (c) preventing or delaying the onset of at least one symptom of the disease.

The terms “polynucleotide,” “nucleotide sequence,” “nucleic acid,” “nucleic acid molecule,” “nucleic acid sequence,” and “oligonucleotide” refer to a series of nucleotide bases (also called “nucleotides”) in DNA and RNA and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. A nucleotide sequence typically carries genetic information, including the information used by cellular machinery to make proteins and enzymes. These terms include double- or single-stranded genomic DNA, RNA, any synthetic and genetically manipulated polynucleotide, and both sense and antisense polynucleotides. This also includes nucleic acids containing modified bases.

Conventional IUPAC notation is used in nucleotide sequences presented herein, as shown in Table 1, below (see also Cornish-Bowden 1985, incorporated by reference herein). It should be noted, however, that “T” denotes “Thymine or Uracil” insofar as a given sequence (such as a gRNA sequence) may be encoded by either DNA or RNA.

TABLE 1
IUPAC nucleic acid notation

| Character | Base |
|---|---|
| A | Adenine |
| T | Thymine |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G, or T/U |
| V | A, C, or G |
| H | A, C, or T/U |
| D | A, G, or T/U |
| N | A, C, G, or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments, variants, derivatives and analogs of such proteins. Peptide sequences are presented using conventional notation, beginning with the amino or N-terminus on the left, and proceeding to the carboxyl or C-terminus on the right. Standard one-letter or three-letter abbreviations may be used.

The term “antigen” as used herein refers to an immunogenic molecule, subunit, or fragment thereof capable of provoking an immune response. This immune response may involve activation of specific immunologically-competent cells, antibody production, or both. An antigen may be, for example, a peptide, glycopeptide, polypeptide, glycopolypeptide, polysaccharide, lipid, or the like. For example, an antigen may be any molecule or a linear molecular fragment such as a peptide, which is derived by processing of the native antigen that can be recognized by a T-cell receptor (TCR). DNA sequences encoding an antigen may be provided. The DNA sequences that encode an antigen can be optimized for expression in a cell. For example, the DNA sequences encoding an antigen may further comprise a promoter upstream of the DNA sequence encoding the antigen, or other heterologous DNA sequences, a transcription termination signal downstream of the sequence encoding the antigen, or both. It will be apparent to one of skill in the art that an antigen can be produced recombinantly, synthesized, or derived from a biological sample. Exemplary biological samples that can contain one or more antigens include, for example, biological fluids, cells, tumor samples, tissue samples, or combinations thereof. Antigens can be produced by one or more cells that have been genetically engineered or modified to express an antigen. The antigens may be optimized for stable transcription or expression by the one or more cells that have been genetically engineered or modified to produce the antigen. For example, the antigens may be codon optimized by techniques including, but not limited to, by altering sequences of four of the same nucleotides in a row (e.g., AAAA, TTTT, GGGG, CCCC) by introducing point mutations that do not result in amino acid changes in the encoded protein, or by adapting codon usage for use in a specific cell species.

The term “epitope” as used herein includes any structure, molecule, amino acid sequence, or protein determinant that is recognized and specifically bound by a cognate binding molecule, for example, a T cell receptor, chimeric antigen receptor, or any other binding domain, molecule, or protein.

The term “single chain variable” or its abbreviation “scFv” as used herein refers to a single-chain variable fragment of an antibody. An scFv is a fusion protein of the variable regions of the heavy and light chains of immunoglobulins, connected by a short linker peptide.

The term “chimeric antigen receptor” (CAR) refers to a CAR of the present disclosure engineered to contain two or more naturally occurring (or engineered) amino acid sequences linked together in a way that does not occur naturally or does not occur naturally in a host cell, which CAR can function as a receptor when present on a surface of a cell. CARs of the present disclosure include an extracellular portion comprising an antigen-binding domain, such as one obtained or derived from an immunoglobulin, such as an scFv derived from an antibody linked to a transmembrane region and one or more intracellular signaling domains (optionally containing co-stimulatory domain(s)) (see, e.g., Sadelain et al., 2013; see also Harris & Kranz, 2016; Stone et al., 2014).

A “T cell” or “T lymphocyte” is an immune system cell that matures in the thymus and produces TCRs, including αβT cells and γδT cells. T cells can be naïve (not exposed to antigen; increased expression of CD62L, CCR7, CD28, CD3, CD127, and CD45RA, and decreased expression of CD45RO as compared to TCM), memory T cells (TM) (antigen-experienced and long-lived), and effector cells (antigen-experienced, cytotoxic). TM can be further divided into subsets of central memory T cells (TCM, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or TCM).

A “natural killer cell” or “NK cell” is an immune system cell, a type of cytotoxic lymphocyte, that is capable of rapidly responding to a wide variety of pathological challenges. NK cells are capable of killing virus-infected cells and detecting and killing stressed cells and tumor cells without requiring any priming or prior activation, i.e., they do not require antibodies and a major histocompatibility complex (MHC) to be presented on the cell surface to kill a cell. This ability allows NK cells to respond faster than other immune cells such as cytotoxic T cells.

A peripheral blood mononuclear cell (PBMC) is a diverse class of immune cells that includes lymphocytes (T cells, B cells, and NK cells), dendritic cells, and monocytes. PBMCs originate from hematopoietic stem cells (HSCs) in the bone marrow and give rise to all of the cells in the immune system. PBMCs refer to any peripheral blood cell that has a round nucleus.

An “MHC protein” as used herein refers to a major histocompatibility complex (MHC) protein. MHC proteins are also called human leukocyte antigens (HLA), or the H2 locus in mice, which are protein molecules that are expressed on the surface of a cell that confer a unique antigenic identity to the cell. MHC/HLA antigens serve as target molecules that are recognized by T-cells and NK cells as being derived from the same source of hematopoietic reconstituting stem cells as the immune effector cells (“self”) or as being derived from a different source of hematopoietic reconstituting cells (“non-self”). Two primary classes of HLA antigens are recognized (HLA class I and HLA class II). The MHC proteins used herein may be from any mammalian or avian species, for example, primates (particularly humans); rodents (including mice, rats, and hamsters); equines, bovines, canines, felines, rabbits etc. Of particular interest are the human HLA proteins and the murine H-2 proteins. Included in the HLA proteins are the class I proteins HLA-A, HLA-B, HLA-C, and 32-microglobulin, and the class II subunits HLA-DPα, HLA-DPβ, HLA-DQα, HLA-DQβ, HLA-DRα and HLA-DRβ. Included in the murine H-2 subunits are the class I H-2K, H-2D, H-2L, and the class II I-Aa, I-Aβ, I-Eα and I-Eβ, and β2-microglobulin. The MHC binding domains are typically a soluble form of the usually membrane-bound protein. The soluble form is derived from the native form by a deletion of the transmembrane domain. Conveniently, the protein is truncated, removing both the cytoplasmic and transmembrane domains. In certain embodiments, the binding domains of an MHC protein are soluble domains of class II α and β chain. In some such embodiments, the binding domains have been subjected to mutagenesis and selected for amino acid changes that enhance the solubility of the single chain polypeptide without altering the peptide binding contacts.

A “T cell receptor” or “TCR” as used herein refers to the antigen/MHC binding heterodimeric protein product of a vertebrate, e.g. mammalian, TCR gene complex, including the human TCR α, β, γ, and δ chains. A TCR can be recombinant, such as a protein product that is not derived from a mammalian TCR gene complex, or naturally-occurring (e.g., derived from a mammalian TCR gene complex).

The term “synTac” as used herein refers to artificial immunological synapse for T-cell activation (synTacs) because they are fabricated to recapitulate the antigen-specific and/or costimulatory, inhibitory or cytokine signals experienced at the immunological synapse. synTacs consist of covalently tethered peptide-Class I MHC modules (c-pMHC), which are further covalently linked to modulatory domain consisting of either costimulatory, inhibitory or cytokine molecules, presented in the context of an Fc domain scaffold.

All antigens, proteins, peptides, targeting moieties, targeted moieties, targets nucleic acids, modulatory domains, and the like recited herein may have at least about 70%, at least about 75%, at least about 80%, at least about 85%, at least about 90%, at least about 95%, at least about 98%, at least about 99%, or 100% sequence homology with the listed sequence, gene, or protein.

### Cell Modulation

The present disclosure is directed to methods for modulating one or more genetically modified cells ex vivo and/or in vivo. The method comprises the steps of: (a) contacting a cell expressing a receptor that recognizes a cognate peptide with a fusion protein, the fusion protein having at least one major histocompatibility complex (MHC) molecule and at least one cognate peptide, the cognate peptide capable of being recognized by the receptor; (b) culturing the cell for a period of time to generate a plurality of cells, the plurality of cells having a receptor that recognizes the cognate peptide; (c) transducing the plurality of cells with a polynucleotide encoding a chimeric antigen receptor (CAR) to generate a plurality of CAR-expressing cells having both the receptor that recognizes the cognate peptide and the CAR; and (d) contacting the plurality of CAR-expressing cells with an antigen-specific fusion protein to modulate the CAR-expressing cells. In some embodiments, the cognate peptide is 6 to 18 amino acid residues in length, i.e., 6, 7, 8, 9, 10, 11, 12, 13, 14, 15, 16, 17 or 18 amino acid residues. In some embodiments, the cognate peptide includes but is not limited to HIV Gag 77-85 (SL9) (SLYNTVATL; SEQ ID NO: 1) or SL9 variant (SLFNTIAVL; SEQ ID NO: 2), HIV Nef 190-198 (AFHHVAREL) (SEQ ID NO: 3), HIV reverse transcriptase 476-484 (ILKEPVHGV; SEQ ID NO: 4), HIV reverse transcriptase 179-187 (VIYQMDDL; SEQ ID NO: 5), CMV pp65 (495-503) (NLVPMVATV; SEQ ID NO: 6), and EBV LMP-2 (426-434) (CLGGLLTMV; SEQ ID NO: 7), or a peptide having an amino acid sequence at least 75%, at least 80%, at least 85%, at least 90%, or at least 95% identical to the sequence of one of the aforementioned peptides.

In certain aspects, the methods provided herein enable the modulation of a genetically modified cell, e.g., CAR-expressing cells (e.g., a CAR-T cell, a CAR-expressing PBMC, a CAR-expressing NK cell, or a CAR-expressing CD8 cell) either ex vivo, e.g., after transduction with the CAR vector or in vivo, e.g., after infusion of the genetically modified cells into a subject.

In step (a), a cell expressing a receptor that recognizes a cognate peptide is contacted with a fusion protein. The fusion protein has at least one major histocompatibility complex (MHC) molecule and at least one cognate peptide, and the cognate peptide is capable of being recognized by the receptor.

In certain embodiments, the cell is a T cell, or a NK cell. In some embodiments, the cell is a CD8+ T cell, a CD4+ T cell, a CD8− CD4− double negative T cell, a γδ T cell, an NK cell, an invariant natural killer T cell (iNKT), or any combination thereof. In some embodiments, the cell is an effector memory T cell, a stem cell memory T cell, a central memory T cell, a naïve T cell, an NK cell, or any combination thereof.

In certain embodiments, the methods disclosed herein may include activating and expanding T cells expressing a specific T cell receptor (TCR) that recognizes a cognate peptide (e.g., peptide, lipid, metabolite, etc.) presented by a specific major histocompatibility complex (MHC) molecule. In certain embodiments, the T cells are selectively activated and/or expanded by treatment with a fusion protein having at least one MHC presenting at least one cognate peptide (e.g., an MHC-peptide-fusion protein) capable of selectively binding to their TCRs. The cognate peptide recognized by the TCR, which can be presented by the fusion protein can be a peptide, lipid, metabolite, carbohydrate, or an immunogenic fragment or epitope thereof. The cognate peptide of the fusion protein can be derived from sequences in the coding region and also from the non-canonical coding regions (e.g., 5′- and 3′-untranslated regions, introns and regulatory sequences in the pathogen genome), as well as from peptide-fusions formed within the proteasome. In short, any peptide, lipid, carbohydrate or metabolite derived from viruses, bacteria, fungi or other micro-organisms or cells by any mechanism.

In certain embodiments, cell activation (e.g., T cell activation) by this fusion protein could be further modulated by linking this fusion protein to a modulatory domain consisting of either a costimulatory binder, coinhibitory binder, cytokine or immunomodulatory molecule (e.g., MHC-peptide-modulatory domain fusion protein).

In step (b), after contacting the cell with the fusion protein, the cell is cultured for a period of time to generate a plurality of cells by proliferation that have a receptor that recognizes the cognate peptide. In certain embodiments, the cells are T cells that are cultured for a period of time to generate a plurality of cognate peptide-specific T cells which have a TCR that recognizes the cognate peptide.

In step (c), the plurality of cells is transduced with a polynucleotide encoding a CAR to generate a plurality of polynucleotide encoding a chimeric antigen receptor (CAR) to generate a plurality of CAR-expressing cells having both the receptor that recognizes an antigen and the CAR. In certain embodiments, the cells are T cells and transducing the plurality of T cells with a polynucleotide encoding a CAR generates a plurality of CAR-expressing cells which in addition to the CAR also express on their surface the receptor that recognizes the cognate peptide. The expression of the receptor by the CAR-expressing cells enables subsequent treatment of these cells, either in vitro or in vivo, as in step (d).

In step (d), the plurality of CAR-expressing cells is contacted with the fusion protein to modulate the CAR-expressing cells. In certain embodiments, this contacting step occurs ex vivo. In other embodiments, the contacting step occurs in vivo. In certain aspects, contacting the plurality of CAR-expressing cells with the fusion protein (e.g., MHC-peptide-fusion protein and/or MHC-peptide-modulatory domain fusion protein) modulates the CAR-expressing cells. In certain embodiments, the fusion protein binds to the receptor alone or, in some embodiments, also in combination with a modulatory domain ligand to selectively modulate the CAR-expressing cells by selective delivery of an activation signal alone from the receptor or in combination with a modulatory signal from the linked costimulatory binder, coinhibitory binder or cytokine, either to selectively activate, expand and differentiate the CAR-expressing cells or suppress the CAR-expressing cells.

In certain embodiments, the activation and expansion of the cells and CAR-expressing cells is accomplished by treatment with a fusion protein (e.g., MHC-peptide-fusion protein and/or MHC-peptide-modulatory domain fusion protein) such as a synTac. In other embodiments, cells expressing a receptor that recognizes a cognate peptide can be treated with other modalities for antigen-specific immune cell activation, modulation and expansion utilizing peptide-MHC modules for selective immune cell targeting including engineered constructs such as a soluble protein scaffold, bead-based nanoparticles or membrane-based nanoparticles displaying single or multiple copies of the peptide-MHC for selective delivery of modulatory functions

In certain embodiments, contacting the plurality of CAR-expressing cells with the MHC-peptide-fusion protein and/or MHC-peptide-modulatory domain fusion protein elicits an in vivo or ex vivo expansion of the plurality of CAR-expressing cells.

In certain embodiments, contacting the plurality of CAR-expressing cells with the MHC-peptide-fusion protein and/or MHC-peptide-modulatory domain fusion protein elicits an in vivo or ex vivo modulation of the plurality of CAR-expressing cells. In some embodiments, modulation of the plurality of CAR-expressing cells may include, for example, inducing proliferation of the CAR-expressing cell, inducing differentiation of the CAR-expressing cell, stimulating the CAR-expressing cell to induce cytotoxic effects, to secrete immune activating cytokines or chemokines or immune inhibiting cytokines of chemokines, inhibiting the CAR-expressing cell, inducing apoptosis or necrosis in the CAR-expressing cell, inducing quiescence, or any combination thereof.

In certain embodiments, the CAR-expressing cell is selected from one of the following: a T cell, an NK cell, or a PBMC.

In certain embodiments, the CAR-expressing cell is a T cell that expresses a particular surface marker or surface markers. For example, the T cell may be a CD4⁺ T cell, a CD8⁺ T cell, a CD4⁻ CD8⁻ double negative T cell, or any suitable T cell.

In certain embodiments, the CAR-expressing cell is an NK cell that expresses a particular surface marker or surface markers. For example, the NK cell may be a CD3⁻ NK cell, a CD56/NCAM1⁺ NK cell, a CD94⁺ NK cell, a CD122/IL-2 R beta NK cell, a CD127/IL-7 R alpha-NK cell, an Fc gamma RIII/CD16^{+/-} NK cell, a KIR family receptor⁺ NK cell, an NKG2A⁺ NK cell, an NKG2D⁺ NK cell, an NKp30⁺ NK cell, an NKp44⁺ NK cell, an NKp46⁺ NK cell, an NKp80⁺ NK cell, a CD11b/Integrin alpha M⁺ NK cell, a CD27⁺ NK cell, a CD161/NK1.1⁺ NK cell, an Integrin alpha 2/CD49b⁺ NK cell, or a Ly49 family receptor⁺ NK cell, or any suitable NK cell.

T cells and NK cells can induce lysis in a target cell via two major pathways. First, T cells may can release cytotoxic granules containing granzymes and perforin via exocytosis. Second, T cells can express a membrane-bound tumor necrosis factor (TNF) family ligand. Upon engaging with their respective receptors, these TNF family ligands can induce apoptosis in the target cell. Additionally, T cells can sensitize stromal cells by secreting cytokines. In certain embodiments, the CAR-expressing cell is stimulated to induce cytotoxic effects in a target cell. In certain embodiments, the CAR-expressing cell secretes a molecule that may be selected from the following: a granzyme, a perforin, a cytokine, a Fas Ligand (FasL), or any appropriate molecule that triggers a cytotoxic effect in the target cell (e.g., lysis or apoptosis).

In certain embodiments, the fusion protein is specific for a eukaryotic pathogen, wherein the cognate peptide of the fusion protein is encoded by a sequence derived from the eukaryotic pathogen. In certain aspects, the eukaryotic pathogen is selected from a virus, a fungus, or a protozoa.

In certain embodiments, the fusion protein is a virus-specific fusion protein, wherein the cognate peptide of the fusion protein is encoded by a sequence derived from a virus. In certain aspects, the virus is selected from the group consisting of a human immunodeficiency virus (HIV), a cytomegalovirus (CMV), an Epstein-Barr virus (EBV), a measles virus, a mumps virus, a varicella virus, an influenza virus, a rotavirus, a polio virus, a hepatitis virus, a diphtheria virus, a pertussis virus, a pneumococcal virus, a meningococcal fusion protein, a zoster virus, a yellow fever virus, a human papillomavirus, a rabies virus, or the like. In some embodiments, the cognate peptide is 6 to 18 amino acid residues in length, i.e., 6, 7, 8, 9, 10, 11, 12, 13, 14, 15, 16, 17, or 18 amino acid residues. In some embodiments, the cognate peptide includes but is not limited to HIV Gag 77-85 (SL9) (SLYNTVATL; SEQ ID NO: 1) or SL9 variant (SLFNTIAVL; SEQ ID NO: 2), HIV Nef 190-198 (AFHHVAREL) (SEQ ID NO: 3), HIV reverse transcriptase 476-484 (ILKEPVHGV; SEQ ID NO: 4), HIV reverse transcriptase 179-187 (VIYQMDDL; SEQ ID NO: 5), CMV pp65 (495-503) (NLVPMVATV; SEQ ID NO: 6), and EBV LMP-2 (426-434) (CLGGLLTMV; SEQ ID NO: 7), or a peptide having an amino acid sequence at least 75%, at least 80%, at least 85%, at least 90%, or at least 95% identical to the sequence of one of the aforementioned peptides.

In certain embodiments, the fusion protein is a fungus-specific fusion protein, wherein the cognate peptide of the fusion protein is encoded by a sequence derived from a fungus. In certain aspects, the fungus is selected from the group consisting of Basidiomycota, Sac fungi, Eomycota, Zygomycota, Chytridiomycota, Microsporidia, Glomeromycota, Hyphomycetes, Neocallimastigomycota, Blastocladiomycota, Kickxellomycotina, Inocybe salicis, Babjeviella inositovra, Aspergillus, Blastomyces, Cryptococccus, Candida, Coccidioides, Histoplasma, Sporothrix, or the like.

In certain embodiments, the fusion protein is a protozoan-specific fusion protein, wherein the cognate peptide of the fusion protein is encoded by a sequence derived from a protozoa. In certain aspects, the protozoa is selected from the group consisting of Leishmania, Cryptosporidium, Balantidium, Toxoplasma gondii, Plasmodium, Balantidium coli, Entamoeba, Entamoeba coli, Entamoeba histolytica, Entamoeba gingivalis, Entamoeba invadens, Entamoeba poleck, Giardia lamblia, Acanthamoeba, Endolimax, Endolimax nana, Iodamoeba, Iodamoeba bütschlii, Archamoebae, Entamoeba moshkovskii, Tritrichomonas, Tritrichomonas foetus, Trichomonas vaginalis, Enteromonas hominis, Entromonas, Entamoebidae, Hartmannella, Entomophthorales, Conidiobolus, Basidiobolus ranarum, Hexamitidae, Amoebida, Retortamonad, Hartminnellidae, Acanthamoebidae, Paramoeba, Conidiobolus incongruus, Conidiobolus coronatus, Malpighamoeba, or the like.

In some embodiments, the fusion protein is a bacteria-specific fusion protein, wherein the cognate peptide of the fusion protein is encoded by a sequence derived from a bacterium. In certain aspects, the bacterium is selected from the group consisting of Escherichia, Escherichia coli, MRSA, Shigella, Salmonella, Pseudomonas, Pseudomonas aeruginosa, Streptococcus, Streptococcus pneumoniae, Asiatic cholera, Listeria monocytogenes, Mycobacterium tuberculosis, Yersinia, Campylobacter, Klebsiella pneumoniae, Entrococcus, Neisseria, Enterobacteriaceae, Group A streptococcus, Legionella pneumophila, Corynebacterium, Staphylococcus aureus, Mycobacterium, Shigella, Helicobacter, O157:H7, Anthrax bacterium, Clostridium botulinum, Salmonella enterica, Yersinia pestis, Clostridium perfringens, Haemophilus influenzae, Rickettsia, Acinetobacter, Campylobacter jejuni, Burkholderia, Haemophilus, Enterococus faecalis, Chlamydia, Bordatella pertussis, Clostridioides difficile, Enterococcus faecium, Klebs-Löffler bacillus, Clostridium tetani, Treponema, Acinetobacter baumanni, Brucella, Treponemia pallidum, Xanthomonas, or the like.

In certain embodiments, the cell may be harvested from a subject. In some embodiments, the cell is a T cell or a natural killer (NK) cell. In some embodiments, the cell is a CD8+ T cell, a CD4+ T cell, a CD8− CD4− double negative T cell, a γδ T cell, an NK cell, an invariant natural killer T cell (iNKT), or any combination thereof. In some embodiments, the cell is an effector memory T cell, a stem cell memory T cell, a central memory T cell, a naïve T cell, an NK cell, or any combination thereof.

In certain embodiments, the subject may be vaccinated, prior to harvesting the cell, with a vaccine containing the cognate peptide or DNA encoding the cognate peptide that is included in the fusion protein. By vaccinating or immunizing the subject with an antigen including the cognate peptide or a DNA construct expressing the antigen including the cognate peptide, the subject should produce a plurality of cells (e.g., T cells) having a receptor that recognizes the cognate peptide (i.e., is specific for the cognate peptide) which can be further expanded by in vitro or in vivo treatment with the fusion protein. In some embodiments, following vaccination or immunization, the plurality of cells specific for the cognate peptide may be harvested from the subject and may be selectively expanded ex vivo by treatment with the fusion protein. In some embodiments, the cognate peptide is 6 to 18 amino acid residues in length, i.e., 6, 7, 8, 9, 10, 11, 12, 13, 14, 15, 16, 17, or 18 amino acid residues. In some embodiments, the cognate peptide includes but is not limited to HIV Gag 77-85 (SL9) (SLYNTVATL; SEQ ID NO: 1) or SL9 variant (SLFNTIAVL; SEQ ID NO: 2), HIV Nef 190-198 (AFHHVAREL) (SEQ ID NO: 3), HIV reverse transcriptase 476-484 (ILKEPVHGV; SEQ ID NO: 4), HIV reverse transcriptase 179-187 (VIYQMDDL; SEQ ID NO: 5), CMV pp65 (495-503) (NLVPMVATV; SEQ ID NO: 6), and EBV LMP-2 (426-434) (CLGGLLTMV; SEQ ID NO: 7), or a peptide having an amino acid sequence at least 75%, at least 80%, at least 85%, at least 90%, or at least 95% identical to the sequence of one of the aforementioned peptides.

In certain embodiments, the vaccine may further comprise a pharmaceutically acceptable excipient. Non-limiting examples of pharmaceutically acceptable excipients include, for example, those described in “Remington: The Science and Practice of Pharmacy”, 19th Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc. In some embodiments, the composition is suitable for administration to a subject, for example, a sterile composition. In some embodiments, the composition is suitable for administration to a human subject, for example, the composition is sterile and is free of detectable pyrogens and/or other toxins.

In some embodiments, the vaccine further comprises other components, such as pharmaceutical grades of mannitol, lactose, starch, magnesium stearate, sodium saccharin, talcum, cellulose, glucose, sucrose, magnesium, carbonate, and the like. In some embodiments, the compositions comprise a pharmaceutically acceptable auxiliary substance as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

In some embodiments, the vaccine may be are in an aqueous solution, powder form, granules, tablets, pills, suppositories, capsules, suspensions, sprays, and the like. The composition may comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions may be prepared using conventional techniques that may include sterile isotonic saline, water, 1,3-butanediol, ethanol, 1,2-propylene glycol, polyglycols mixed with water, Ringer's solution, etc. In one aspect, a coloring agent is added to facilitate in locating and properly placing the composition to the intended treatment site.

Compositions may include a preservative and/or a stabilizer. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorbutanol, 2-ethoxyethanol, and imidurea.

To control tonicity, the composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8, e.g., between 6.5 and 7.5, or between 7.0 and 7.8.

The composition can be administered by any appropriate route, which will be apparent to the skilled person depending on the disease or condition to be treated. Typical routes of administration include intravenous, intra-arterial, intramuscular, subcutaneous, intracranial, intranasal or intraperitoneal.

In some embodiments, the composition may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

In certain embodiments, the plurality of cells specific for the cognate peptide may be expanded to >10% of the total cell population and frequently >50% of the total cell population, which can be further selectively expanded by in vitro or in vivo treatment with fusion protein, may be further enriched to >90% purity by immunomagnetic or flow cytometric sorting after incubation with the appropriate peptide:MHC tetramer prior to transduction with the CAR polynucleotide. In some embodiments, the plurality of cells specific for the cognate peptide is >10%, >20%, >30%, >40%, >50%, >60%, >70%, >80%, or >90% of the total cell population.

In certain embodiments, at least one of the plurality of CAR-expressing cells may be administered to a subject. In some embodiments, the CAR-expressing cell may be an autologous CAR-expressing cell expressing a CAR specific for an antigen that is present on the surface of tumor cells or infected cells. In some embodiments, the CAR-expressing cell may be an allogeneic CAR-expressing cell expressing a chimeric receptor specific for an antigen that is present on the surface of tumor cells such as CD19 or pathogen infected cells such as HIV gp120 envelope protein. In some embodiments, the fusion protein may be administered to the subject to elicit in vivo modulation of the CAR-expressing cell.

In certain embodiments, the in vivo modulation of the CAR-expressing cell induces proliferation of the CAR-expressing cell, induces differentiation of the CAR-expressing cell, stimulates the CAR-expressing cell to induce cytotoxic effects, inhibits the CAR-expressing cell, induces apoptosis or necrosis in the CAR-expressing cell, induces quiescence, or any combination thereof.

In certain embodiments, the fusion protein may further include an additional moiety, wherein the additional moiety comprises a modulatory domain, a co-stimulatory ligand, a co-stimulatory receptor recruitment molecule, a cytokine, a modulatory cytokine, a cytokine receptor engager, a co-inhibitory ligand, a co-inhibitory receptor recruitment molecule, an affinity reagent, or any combination thereof.

In some embodiments, the co-stimulatory or co-inhibitory receptor recruitment molecule may be a single chain Fv (scFv) molecule or other ligand affinity reagent such as a nanobody or an aptamer (peptide, RNA, DNA, modified nucleic acids). In some embodiments, the co-stimulatory receptor recruitment molecule may recognize CD28, 4-1BB, GITR, CD27, ICOS, CD40L, TIM3, OX-40, PD-L1, CTLA4, B7-H1, FasL, PD-1, or the like.

In some embodiments, the cytokine may be IL-2, IL-4, IL-6, IL-7, IL-10, IL-12, IL-15, IL-17, IL-21, IL-23, IFN-gamma, TGF-beta, or the like.

In some embodiments, the affinity reagent capable of binding to the co-stimulatory or co-inhibitory receptor may be B7-1, B7-2, B7-H2, CD40, B7-H1, PD1-1, PDL-2, 4-1BBL, GITRL, CD70, ICOS-L, OX-40L, Fas, or the like.

In certain embodiments, the modulation of the CAR-expressing cell may be via direct binding of the cognate peptide recognized by the receptor of the CAR-expressing cell.

In certain embodiments, the subject may have a disease associated with expression of a tumor antigen, a disease associated with a pathogen, or an autoimmune disease or disorder.

In some embodiments, the disease associated with expression of a tumor antigen may be a proliferative disease, a precancerous condition, a cancer, or a non-cancer related indication associated with expression of the tumor antigen.

In some embodiments, the cancer may be chronic lymphocytic leukemia (CLL), acute leukemia, acute lymphoid leukemia (ALL), B-cell acute lymphoid leukemia (B-ALL), T-cell acute lymphoid leukemia (T-ALL), chronic myelogenous leukemia (CIVIL), B cell prolymphocytic leukemia, blastic plasmacytoid dendritic cell neoplasm, Burkitt's lymphoma, diffuse large B cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia.

In some embodiments, the cancer may be selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of the cancers, and metastatic lesions of the cancers.

In some embodiments, the cancer may be leukemia or lymphoma. In some embodiments, the lymphoma may be lymphoblastic lymphoma or B-cell Non-Hodgkin's lymphoma.

In some embodiments, the pathogen may be a eukaryotic pathogen. In some embodiments, the eukaryotic pathogen is selected from a fungus, or a protozoa.

In some embodiments, the pathogen may be a virus. In some embodiments, the virus may be selected from the group consisting of a human immunodeficiency virus (HIV), a cytomegalovirus (CMV) (e.g., CMV-pp65), an Epstein-Barr virus (EBV), a measles virus, a mumps virus, a varicella virus, an influenza virus, a rotavirus, a polio virus, a hepatitis virus, a diphtheria virus, a pertussis virus, a pneumococcal virus, a meningococcal fusion polypeptide, a zoster virus, a yellow fever virus, a human papillomavirus, a rabies virus, or the like.

In certain embodiments, eukaryotic pathogen may be a fungus. In some embodiments, the fungus may be selected from the group consisting of Basidiomycota, Sac fungi, Eomycota, Zygomycota, Chytridiomycota, Microsporidia, Glomeromycota, Hyphomycetes, Neocallimastigomycota, Blastocladiomycota, Kickxellomycotina, Inocybe salicis, Babjeviella inositovra, Aspergillus, Blastomyces, Cryptococccus, Candida, Coccidioides, Histoplasma, Sporothrix or the like.

In certain embodiments, the eukaryotic pathogen may be a protozoa. In some embodiments, protozoa may be selected from the group consisting of Leishmania, Cryptosporidium, Balantidium, Toxoplasma gondii, Plasmodium, Balantidium coli, Entamoeba, Entamoeba coli, Entamoeba histolytica, Entamoeba gingivalis, Entamoeba invadens, Entamoeba poleck, Giardia lamblia, Acanthamoeba, Endolimax, Endolimax nana, Iodamoeba, Iodamoeba bütschlii, Archamoebae, Entamoeba moshkovskii, Tritrichomonas, Tritrichomonas foetus, Trichomonas vaginalis, Enteromonas hominis, Entromonas, Entamoebidae, Hartmannella, Entomophthorales, Conidiobolus, Basidiobolus ranarum, Hexamitidae, Amoebida, Retortamonad, Hartminnellidae, Acanthamoebidae, Paramoeba, Conidiobolus incongruus, Conidiobolus coronatus, Malpighamoeba, or the like.

In certain embodiments, pathogen may be a bacterial pathogen. In some embodiments, the bacterial pathogen may be selected from the group consisting of Escherichia, Escherichia coli, MRSA, Shigella, Salmonella, Pseudomonas, Pseudomonas aeruginosa, Streptococcus, Streptococcus pneumoniae, Asiatic cholera, Listeria monocytogenes, Mycobacterium tuberculosis, Yersinia, Campylobacter, Klebsiella pneumoniae, Entrococcus, Neisseria, Enterobacteriaceae, Group A streptococcus, Legionella pneumophila, Corynebacterium, Staphylococcus aureus, Mycobacterium, Shigella, Helicobacter, O157:H7, Anthrax bacterium, Clostridium botulinum, Salmonella enterica, Yersinia pestis, Clostridium perfringens, Haemophilus influenzae, Rickettsia, Acinetobacter, Campylobacter jejuni, Burkholderia, Haemophilus, Enterococus faecalis, Chlamydia, Bordatella pertussis, Clostridioides difficile, Enterococcus faecium, Klebs-Löffler bacillus, Clostridium tetani, Treponema, Acinetobacter baumanni, Brucella, Treponemia pallidum, Xanthomonas, or the like.

In certain embodiments, the treatment of patients exhibiting CAR-T cell-associated toxicity with the MHC-peptide-modulatory domain fusion protein will reduce or eliminate an inflammatory response, a cytokine storm, or at least one off-target effect, or any combination thereof as compared to a control treatment.

In certain embodiments, treatment of patients exhibiting CAR-T cell-associated toxicity with the MHC-peptide-modulatory domain fusion protein will reduce the symptom or a side effect experienced by the subject as compared to a control treatment.

In some embodiments, the symptom may be cytokine-release syndrome (CRS), a neurologic toxicity, B-cell aplasia, tumor lysis syndrome (TLS), anaphylaxis, fever, joint/muscle aches, shortness of breath, low blood pressure, confusion, or a seizure.

From the foregoing, it will be appreciated that specific embodiments of the invention have been described herein for purposes of illustration, but that various modifications may be made without deviating from the scope of the invention. Accordingly, the invention is not limited except as by the appended claims.

### EXAMPLES

### Example 1

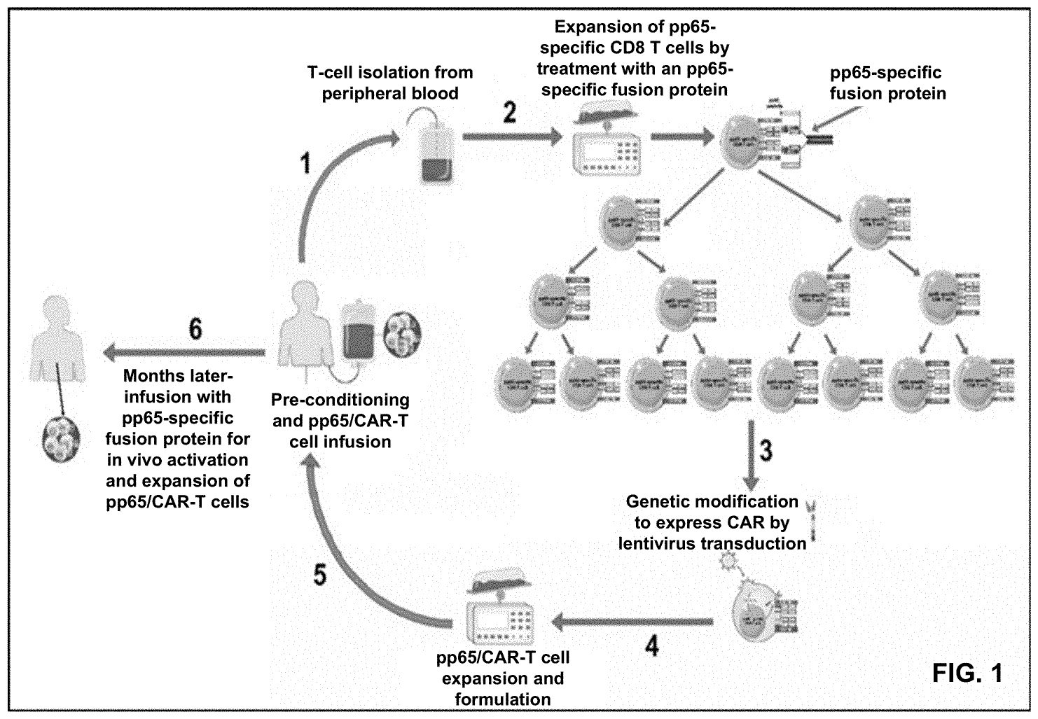

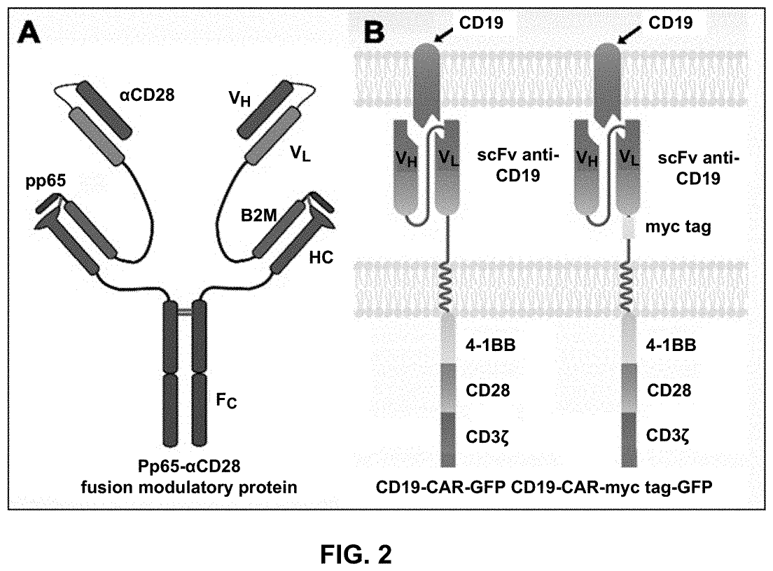

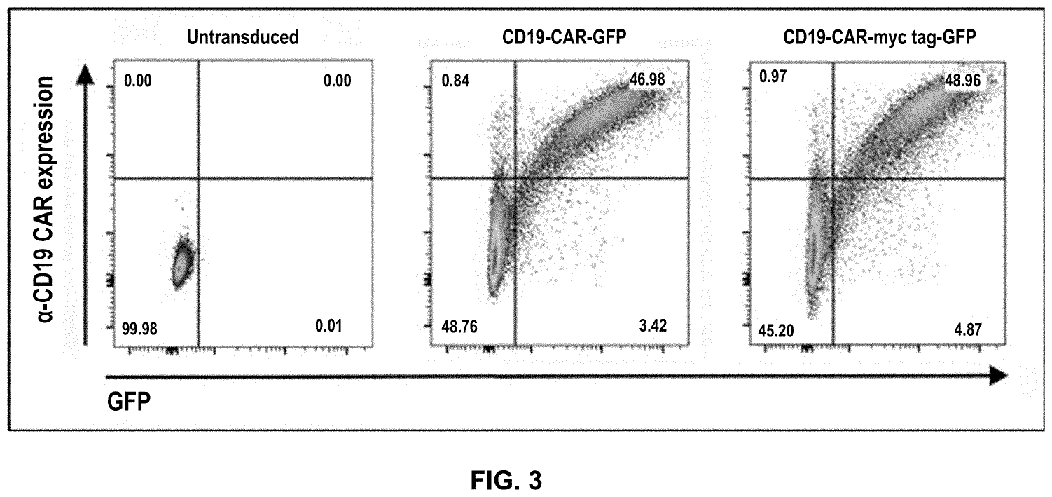

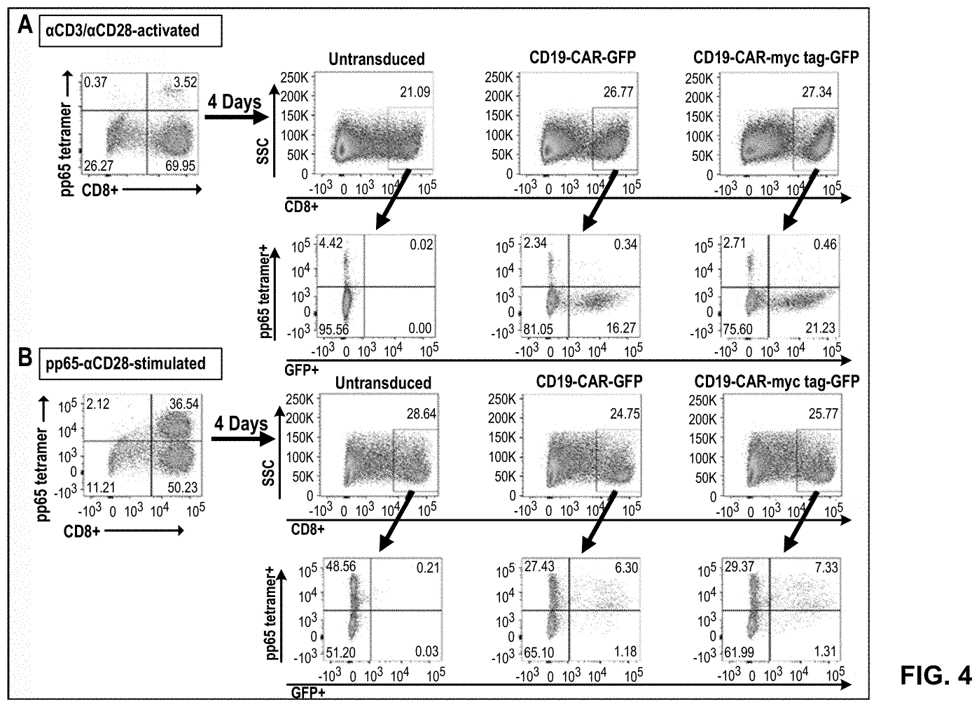

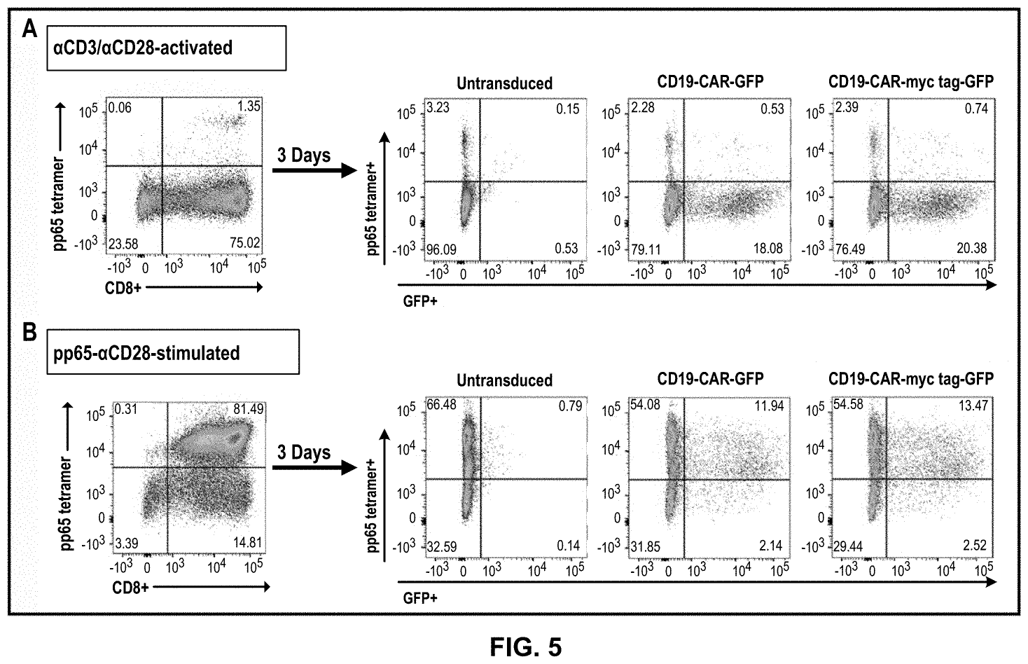

Provided herein are novel methods of modulating CAR-expressing cell function and activity either ex vivo before infusion or in vivo after infusion into patients. An overview of one such method according to the present disclosure is detailed in FIG. 1. In this example, the pp65 CMV-derived epitope is used as an illustrative example of an WIC-peptide-modulatory domain fusion protein. Step 1: T cells were isolated from the peripheral blood of a patient, either by large volume bleed or leukapheresis. Step 2: The T cells were treated with a pp65-specific fusion modulatory protein consisting of a major histocompatibility complex (WIC) molecule (HLA-A*0201) presenting the pp65 peptide (amino acids ^{(495NLVPMVATV503) (SEQ ID NO. 6),}) which is capable of being recognized by the TCRs of the small fraction of pp65-specific T cells present in the peripheral blood and a linked costimulatory binding protein, an scFv specific for CD28 (FIG. 2A). Twelve days after treatment with this pp65-specific fusion modulatory protein, the fraction of pp65-specific T cells markedly expanded to greater than half of the total CD8 T cells (see FIG. 3B). Step 3: These cells were transduced with a lentiviral vector expressing a CAR. Step 4: As needed, the CAR-transduced T cells could be further expanded by ex vivo treatment with the pp65-specific fusion modulatory protein (see FIGS. 4 and 5). Step 5: The patient is then infused with the CAR-T cells, the majority of which also express the pp65-specific TCR. Step 6: Expression of the pp65-specific TCR by the CAR-T cells enables the patient to be subsequently treated by infusion of pp65-specific fusion modulatory proteins linked to either costimulatory molecule binders or cytokines to stimulate the activity/expansion of the CAR-T cells, or infusion of coinhibitory-bearing fusion modulatory protein to inhibit or inactivate the CAR-T cells.

Because efficient transduction of T cells with lentivirus encoding proteins, such as CARs, requires activation of the cells (e.g., CAR-T cells), the standard approach to generate CAR-T cells is to activate the T cells with polyclonal activators such as αCD3 and αCD28 and then transduce them with the lentivirus expressing the CAR. This approach results in the random generation of CAR-T cells expressing TCRs specific for thousands of distinct peptide antigens. A key novel component of the present disclosure is the treatment of a cell or plurality of cells—e.g., PBMCs, NK cells, or memory CD8 cells, or T cells—with a fusion protein e.g., WIC-peptide-fusion protein and/or WIC-peptide-modulatory domain fusion protein—in order to (1) selectively expand the cells specific for the peptide and (2) to make this particular population of cells (e.g., PBMCs, memory CD8 cells, NK cells, or T cells) selectively susceptible to transduction by a CAR lentivirus. As proof of concept, the inventors used two CAR constructs specific for CD19, a scFv specific for CD19 with a GFP reporter or a scFv specific for CD19 with a GFP reporter and an incorporated myc sequence tag (FIG. 2B). Staining with a CD19 detection reagent and flow cytometric analysis demonstrated that expression of the GFP reporter gene correlates with expression of the scFv specific for CD19 and CAR by the CAR-T cells (FIG. 3). Inventors demonstrated that treatment of PBMCs from a donor with a MHC-peptide-modulatory domain fusion protein will selectively activate and expand T cells specific for that peptide and enable the generation of CAR-T cells expressing TCRs specific for that peptide by comparing the TCR peptide specificity of CAR-T cells generated after the standard polyclonal activation with α-CD3 and α-CD28 to those generated after stimulation with a MHC-peptide-modulatory domain fusion protein such as the pp65-αCD28. Three days after PBMC containing T cells were stimulated with αCD3 and αCD28 to activate the T cells, the cells were either untransduced or transduced with a lentivirus expressing CD19-CAR with a GFP reporter or a CD19-CAR with a GFP and an incorporated myc tag and then stained with the pp65 tetramer (FIG. 4A, lower panels). Based on GFP expression, the fraction of CD8+ T cell transduced by the CD19-CAR-GFP reporter lentiviral vector or the CD19-CAR-myc tag-GFP lentiviral vector was 16% and 21%, respectively, and most importantly, <1% bound the pp65 tetramer+ CD8 cells. In contrast, when PBMC were treated with a pp65-αCD28 fusion protein, the pp65-specific CD8 T cell population was selectively expanded by greater than 10-fold to >40% of the total CD8 T cell population and generated CAR-T cells of which almost all expressed a pp65-specific TCR with almost 20% of the pp65-specific CD8 T cells genetically engineered into CD19-specific CAR-T cells as compared to <2% of the global T cell population after gating on the CD8+ T cells (FIG. 4B, lower panels) for the expression of the GFP-reporter and CD8+ T cell population. Further culture of the cells after treatment with the pp65-αCD28 fusion protein for an additional 5 days further expanded the pp65-specific CD8 T cell population by greater than 60-fold to >80% of the total CD8 T cell population and generated CAR-T cells of which almost all expressed a pp65-specific TCR with almost 20% of the pp65-specific CD8 T cells genetically engineered into CD19-specific CAR-T cells (FIGS. 5A, 5B). After infusion of these cells into patients, the CD19-specific CAR-T cells would be responsive to modulation by pp65-specific fusion proteins linked either to stimulatory domains for subsequent in vivo activation and expansion and to inhibitory domains for subsequent in vivo suppression and elimination.

This example demonstrates that the treatment of PBMCs from a donor with pp65-αCD28 synTac fusion protein, an WIC-peptide-modulatory domain fusion protein specific for a CMV-derived peptide (pp65-NLVPMVATV), selectively activated and expanded T cells specific for that peptide, conferred them with susceptibility to transduction by CAR-expressing lentivirus and generate CAR-T cells expressing TCRs specific for that pp65 peptide. This result was accomplished by comparing the TCR peptide specificity of CAR-T cells generated after the standard polyclonal activation with α-CD3 and α-CD28 to those generated after stimulation with an WIC-peptide-modulatory domain fusion protein such as the pp65-αCD28 synTac (FIGS. 4 and 5).

### Example 2

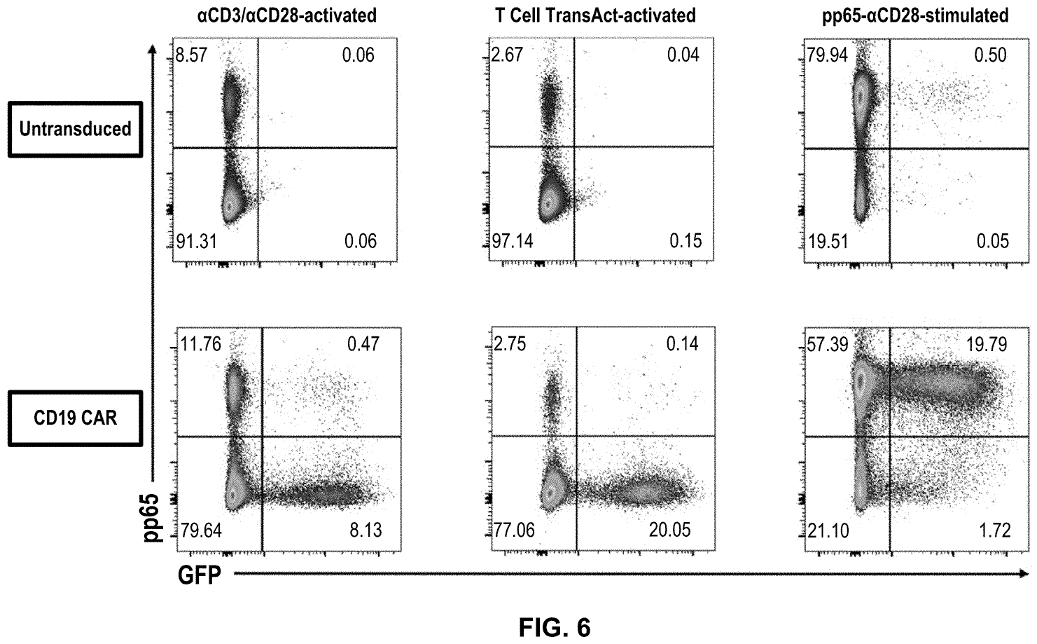

The data in Example 1 was recapitulated and the results are shown in FIG. 6, which demonstrates that pp65-αCD28 synTac treatment selectively activated CMV-specific CD8+ T cells to enable their specific conversion into CD19 CAR T-cells. CD8+ T cells were purified from PBMC and then treated with either soluble αCD3/αCD28 or αCD3/αCD28-conjugated T Cell TransAct nanomatrix for 3 days or a pp65-αCD28 synTac (0.1 nM) for 7 days. The activated cells were either untransduced or transduced with a lentivirus expressing CD19-CAR with a GFP reporter. Three days later the cells were analyzed by flow cytometry for expression of CD8 and then the CD8+ population was gated and examined for the fraction of cells binding the pp65 tetramer and expressing GFP, which is a surrogate for expression of the CAR, are shown. In contrast to the CD8+ T cells stimulated with soluble αCD3/αCD28 or αCD3/αCD28-conjugated T Cell TransAct nanomatrix where all the CAR-T cells (indicated by GFP) were in the pp65 negative population, in the CD8+ T cells stimulated with pp65-αCD28 synTac, almost all of the CAR-T cells (indicated by GFP) were in the pp65 positive population. In addition, the fraction of transduced CAR-T cells was equivalent for the αCD3/αCD28-conjugated T Cell TransAct nanomatrix-stimulated CD8+ T cells as compared to the pp65-αCD28 synTac-treated CD8+ T cells. Thus, treatment with pp65-αCD28 synTac enabled the selective generation of CAR-T cells that were also CMV (pp65)-specific.

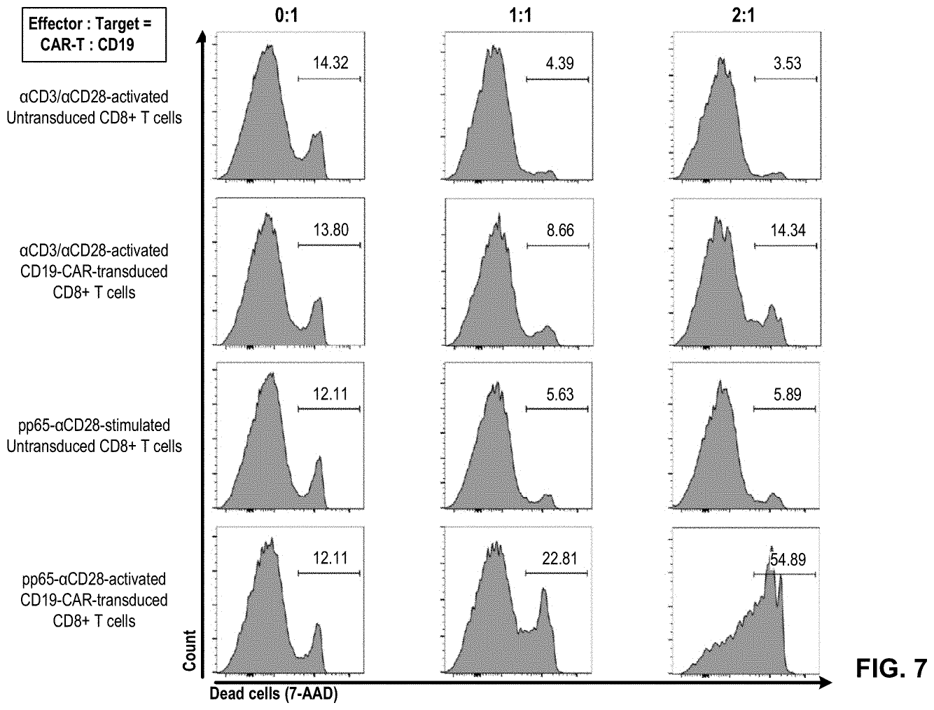

CAR-T cells derived from virus-specific CD8+ T cells, which represent memory effector cells generated after a virus infection and capable of rapid expansion and cytotoxic activity after reinfection, could display more potent CAR-T cell cytotoxic activity than CAR-T cells derived from polyclonal activation of all CD8+ T cells by transduction after standard αCD3/αCD28 activation. For this purpose, studies were carried out to investigate whether the CMV-pp65-specific CD8+ T cells converted into CD19-specific CAR-T cells by transduction with the CAR lentivirus after selective activation of pp65-specific CD8+ T cells by pp65-αCD28 synTac treatment displayed more potent cytotoxic activity against B cells than CAR-T cells generated by transducing CD8+ T cells after global standard activation with αCD3/αCD28 or αCD3/αCD28-conjugated T Cell TransAct nanomatrix-stimulated CD8+ T cells. After generation of these different CAR-T cells, the potency of their activity was determined by incubating the CAR-T cells with purified B cells which express CD19 followed by quantification of the fraction of B cells killed by the CD19-specific CAR-T cells. As demonstrated in FIG. 7, the pp65-αCD28 synTac-activated CD19 CAR T-cells displayed more potent killing of purified CD19 cells in vitro than CAR T-cells activated by standard αCD3/αCD28 activation. At an E:T ratio of 2:1, the pp65-αCD28 synTac)-activated CD19 CAR T-cells killed ˜55% of the target B cells, which was almost 4-fold higher than the fraction of B cells (˜14%) killed by the CAR T-cells activated by standard αCD3/αCD28 activation.

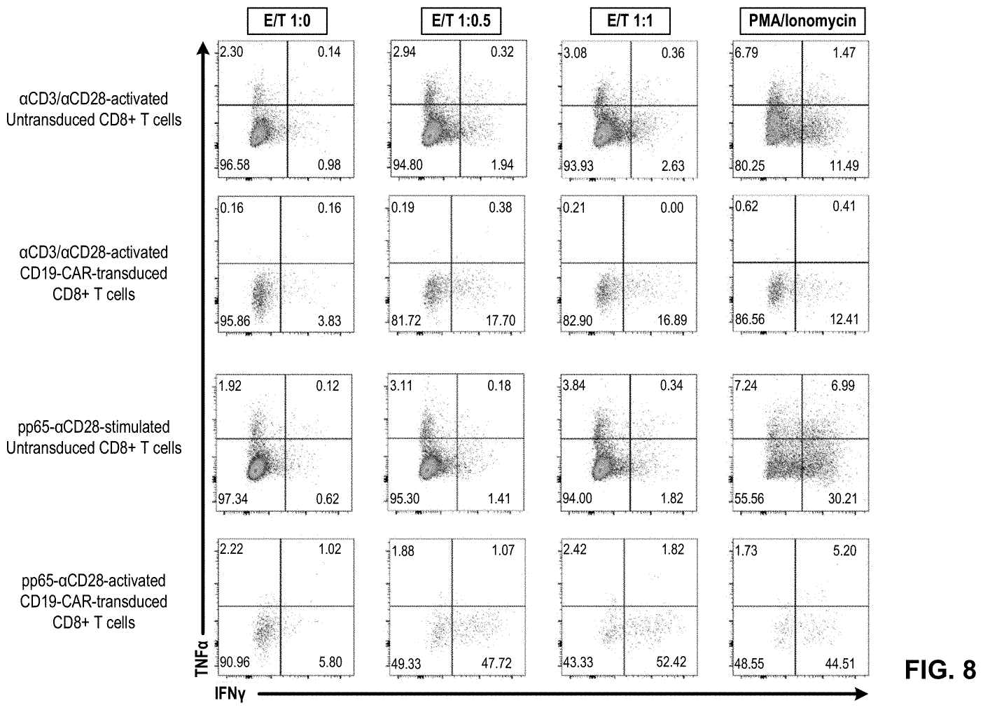

To evaluate further the functional activity of CAR-T cells generated after selective activation of pp65-specific CD8+ T cells by pp65-αCD28 synTac as compared to CAR-T cells generated by standard αCD3/αCD28, the relative response of the CAR-T cells after activation of their αCD19 CAR was evaluated by measuring IFN-γ and TNF-α production after binding to their target CD19 expressed by B cells. The CAR-T cells were co-cultured with purified B cells and analyzed for intracellular production of IFN-γ and TNF-α by flow cytometric analysis. As shown in FIG. 8, minimal IFN-γ was produced by all of the CAR-T cells in the absence of target B cells or by untransduced CD8+ T cells in the absence and presence of B cells. In contrast, after incubation with B cells at an E:T ratio of 1:1 for 1 day, over 50% of the pp65-αCD28 synTac-treated CD19 CAR T-cells expressed IFN-γ. This is about 3-fold greater than the about 17% of the IFN-γ producing CAR T-cells generated by standard αCD3/αCD28 activation observed after 1 day of incubation with purified B cells at an equivalent 1:1 E:t ratio. Taken together, these data provide further support for the proposition that generation of CAR-T cells from virus-specific CD8+ T cells by stimulation with virus-specific synTac can generate CAR-T cells with more potent killing activity associated with more effective elimination of leukemia and cancer cells.

What is claimed is:

1. A method of modulating one or more genetically modified cells ex vivo and/or in vivo, comprising:
contacting a cell expressing a receptor that recognizes a cognate peptide with a fusion protein, the fusion protein having at least one major histocompatibility complex (MEW) molecule and at least one cognate peptide capable of being recognized by the receptor;
culturing the cell for a period of time to generate a plurality of cells, the plurality of cells having the receptor that recognizes the cognate peptide;
transducing the plurality of cells with a polynucleotide encoding a chimeric antigen receptor (CAR) to generate a plurality of CAR-expressing cells having both the receptor that recognizes the cognate peptide and the CAR; and
contacting the plurality of CAR-expressing cells with the fusion protein to modulate the CAR-expressing cells.

2. The method of claim 1, wherein the cell is selected from a T cell, a natural killer (NK) cell, a memory CD8 cell, or a peripheral blood mononuclear cell (PBMC).

3. The method of claim 1, wherein the fusion protein is a synTac.

4. The method of either one of claims 1-3, where contacting the plurality of CAR-expressing cells with the fusion protein elicits an in vivo or ex vivo expansion or modulation of the plurality of CAR-expressing cells.

5. The method of any one of claims 1-4, wherein modulating the CAR-expressing cell induces proliferation of the CAR-expressing cell, induces differentiation of the CAR-expressing cell, stimulates the CAR-expressing cell to induce cytotoxic effects, inhibits the CAR-expressing cell, induces apoptosis or necrosis in the CAR-expressing cell, induces quiescence, or any combination thereof.

6. The method of any one of claims 1-5, wherein the CAR-expressing cell is selected from one of the following: a T cell, an NK cell, or a PBMC.

7. The method of claim 6, wherein the CAR-expressing cell is a T cell, wherein the T cell is selected from one of the following: a CD4⁺ T cell, a CD8⁺ T cell, a CD4⁻ CD8⁻ double negative T cell.

8. The method of claim 7, wherein the CAR-expressing cell is an NK cell, wherein the NK cell is selected from one of the following: a CD3⁻ NK cell, a CD56/NCAM1⁺ NK cell, a CD94⁺ NK cell, a CD122/IL-2 R beta⁺ NK cell, a CD127/IL-7 R alpha-NK cell, an Fc gamma RIII/CD16^{+/-} NK cell, a KIR family receptor⁺ NK cell, an NKG2A⁺ NK cell, an NKG2D⁺ NK cell, an NKp30⁺ NK cell, an NKp44⁺ NK cell, an NKp46⁺ NK cell, an NKp80⁺ NK cell, a CD11b/Integrin alpha M⁺ NK cell, a CD27⁺ NK cell, a CD161/NK1.1⁺ NK cell, an Integrin alpha 2/CD49b⁺ NK cell, or a Ly49 family receptor⁺ NK cell.

9. The method of claim 5, wherein the CAR-expressing cell is stimulated to induce cytotoxic effects in a target cell, and wherein the CAR-expressing cell secretes a molecule selected from the following: a granzyme, a perforin, a cytokine, a Fas Ligand (FasL).

10. The method of claim 9, wherein the target cell is a tumor cell.

11. The method of any one of claims 1-10, wherein the fusion protein is specific for a eukaryotic pathogen, wherein the cognate peptide of the fusion protein is encoded by a sequence derived from the eukaryotic pathogen.

12. The method of claim 5, wherein the eukaryotic pathogen is selected from a virus, a fungus, or a protozoa.

13. The method of any one of claims 1-12, wherein the fusion protein is a virus-specific fusion protein, wherein the cognate peptide of the fusion protein is encoded by a sequence derived from a virus.

14. The method of claim 13, wherein the virus is selected from the group consisting of a human immunodeficiency virus (HIV), a cytomegalovirus (CMV), an Epstein-Barr virus (EBV), a measles virus, a mumps virus, a varicella virus, an influenza virus, a rotavirus, a polio virus, a hepatitis virus, a diphtheria virus, a pertussis virus, a pneumococcal virus, a meningococcal fusion protein, a zoster virus, a yellow fever virus, a human papillomavirus, a rabies virus, or the like.

15. The method of any one of claims 1-11, wherein the fusion protein is a fungus-specific fusion protein, wherein the cognate peptide of the fusion protein is encoded by a sequence derived from a fungus.

16. The method of claim 15, wherein the fungus is selected from the group consisting of Basidiomycota, Sac fungi, Eomycota, Zygomycota, Chytridiomycota, Microsporidia, Glomeromycota, Hyphomycetes, Neocallimastigomycota, Blastocladiomycota, Kickxellomycotina, Inocybe salicis, Babjeviella inositovra, Aspergillus, Blastomyces, Cryptococccus, Candida, Coccidioides, Histoplasma, and Sporothrix.

17. The method of any one of claims 1-11, wherein the fusion protein is a protozoan-specific fusion protein, wherein the cognate peptide of the fusion protein is encoded by a sequence derived from a protozoa.

18. The method of claim 17, wherein the protozoa is selected from the group consisting of Leishmania, Cryptosporidium, Balantidium, Toxoplasma gondii, Plasmodium, Balantidium coli, Entamoeba, Entamoeba coli, Entamoeba histolytica, Entamoeba gingivalis, Entamoeba invadens, Entamoeba poleck, Giardia lamblia, Acanthamoeba, Endolimax, Endolimax nana, Iodamoeba, Iodamoeba bütschlii, Archamoebae, Entamoeba moshkovskii, Tritrichomonas, Tritrichomonas foetus, Trichomonas vaginalis, Enteromonas hominis, Entromonas, Entamoebidae, Hartmannella, Entomophthorales, Conidiobolus, Basidiobolus ranarum, Hexamitidae, Amoebida, Retortamonad, Hartminnellidae, Acanthamoebidae, Paramoeba, Conidiobolus incongruus, Conidiobolus coronatus, and Malpighamoeba.

19. The method of any one of claims 1-10, wherein the fusion protein is a bacteria-specific fusion protein, wherein the cognate peptide of the fusion protein is encoded by a sequence derived from a bacterium.

20. The method of claim 19, wherein the bacterium is selected from the group consisting of Escherichia, Escherichia coli, MRSA, Shigella, Salmonella, Pseudomonas, Pseudomonas aeruginosa, Streptococcus, Streptococcus pneumoniae, Asiatic cholera, Listeria monocytogenes, Mycobacterium tuberculosis, Yersinia, Campylobacter, Klebsiella pneumoniae, Entrococcus, Neisseria, Enterobacteriaceae, Group A streptococcus, Legionella pneumophila, Corynebacterium, Staphylococcus aureus, Mycobacterium, Shigella, Helicobacter, O157:H7, Anthrax bacterium, Clostridium botulinum, Salmonella enterica, Yersinia pestis, Clostridium perfringens, Haemophilus influenzae, Rickettsia, Acinetobacter, Campylobacter jejuni, Burkholderia, Haemophilus, Enterococus faecalis, Chlamydia, Bordatella pertussis, Clostridioides difficile, Enterococcus faecium, Klebs-Löffler bacillus, Clostridium tetani, Treponema, Acinetobacter baumanni, Brucella, Treponemia pallidum, and Xanthomonas.

21. The method of any one of claims 1-20, further comprising harvesting the cell from a subject.

22. The method of claim 21, further comprising vaccinating the subject with a vaccine containing the cognate peptide recognized by the receptor prior to harvesting the cell.

23. The method of any one of claims 1-22, further comprising purifying the plurality of CAR-expressing cells from a population of cells.

24. The method of any one of claims 1-23, further comprising administering at least one of the plurality of CAR-expressing cells to a subject.

25. The method of claim 24, wherein the CAR-expressing cell is an autologous cell or an allogeneic cell.

26. The method of either one of claim 24 or claim 25, further comprising administering to the subject the fusion protein after infusion of the CAR-expressing cell to elicit in vivo modulation of the CAR-expressing cell.

27. The method of claim 26, wherein the in vivo modulation of the CAR-expressing cell induces proliferation of the CAR-expressing cell, induces differentiation of the CAR-expressing cell, stimulates the CAR-expressing cell to induce cytotoxic effects, inhibits the CAR-expressing cell, induces apoptosis or necrosis in the CAR-expressing cell, induces quiescence, or any combination thereof.

28. The method of claim 1 or claim 27, wherein the fusion protein further comprises an additional moiety, wherein the additional moiety comprises a modulatory domain, a co-stimulatory ligand, a co-stimulatory receptor recruitment molecule, a cytokine, a modulatory cytokine, a cytokine receptor engager, a co-inhibitory ligand, a co-inhibitory receptor recruitment molecule, an affinity reagent, or any combination thereof.

29. The method of claim 28, wherein the co-stimulatory or co-inhibitory receptor recruitment molecule is a single chain Fv (scFv) molecule.

30. The method of claim 29, wherein the scFv molecule is capable of binding a co-stimulatory receptor recruitment molecule or co-inhibitory receptor recruitment molecule selected from the group consisting of an scFv molecule recognizing CD28, 4-1BB, GITR, CD27, ICOS, CD40L, TIM3, OX-40, PD-L1, CTLA4, B7-H1, FasL, PD-1, or the like.

31. The method of claim 28, wherein the co-stimulatory receptor recruitment molecule or co-inhibitory recruitment molecule is a ligand or an affinity molecule.

32. The method of claim 31, wherein the ligand or affinity molecule is selected from the group consisting B7-1, B7-2, B7-H2, CD40, B7-H1, PD1-1, PDL-2, 4-1BBL, GITRL, CD70, ICOS-L, OX-40L, Fas, or the like.

33. The method of claim 28, wherein the cytokine is selected from the group consisting IL-2, IL-4, IL-6, IL-7, IL-10, IL-12, IL-15, IL-17, IL-21, IL-23, IFN-gamma, TGF-beta, or the like.

34. The method of any one of claims 26-33, wherein the modulation of the CAR-expressing cell is via direct binding of the CAR-expressing cell.

35. The method of claim 34, wherein the modulation of the CAR-expressing cell induces proliferation of the CAR-expressing cell, induces differentiation of the CAR-expressing cell, stimulates the CAR-expressing cell to induce cytotoxic effects, inhibits the CAR-expressing cell, induces apoptosis or necrosis in the CAR-expressing cell, induces quiescence, or any combination thereof.

36. The method of any one of claims 26-35, wherein the subject has a disease associated with expression of a tumor antigen, a disease associated with a pathogen, or an autoimmune disease or disorder.

37. The method of claim 26, wherein the disease associated with expression of a tumor antigen is a proliferative disease, a precancerous condition, a cancer, or a non-cancer related indication associated with expression of the tumor antigen.

38. The method of claim 37, wherein the cancer is selected from the group consisting of chronic lymphocytic leukemia (CLL), acute leukemia, acute lymphoid leukemia (ALL), B-cell acute lymphoid leukemia (B-ALL), T-cell acute lymphoid leukemia (T-ALL), chronic myelogenous leukemia (CIVIL), B cell prolymphocytic leukemia, blastic plasmacytoid dendritic cell neoplasm, Burkitt's lymphoma, diffuse large B cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia.

39. The method of claim 37, wherein the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of the cancers, and metastatic lesions of the cancers.

40. The method of claim 37, wherein the cancer is leukemia or lymphoma.

41. The method of claim 40, wherein the lymphoma is lymphoblastic lymphoma or B-cell Non-Hodgkin's lymphoma.

42. The method of claim 36, wherein the pathogen is a eukaryotic pathogen.

43. The method of claim 42, wherein the eukaryotic pathogen is selected from a virus, a fungus, or a protozoa.

44. The method of claim 43, wherein the eukaryotic pathogen is a virus.

45. The method of claim 44, wherein the virus is selected from the group consisting of a human immunodeficiency virus (HIV), a cytomegalovirus (CMV) (e.g., CMV-pp65), an Epstein-Barr virus (EBV), a measles virus, a mumps virus, a varicella virus, an influenza virus, a rotavirus, a polio virus, a hepatitis virus, a diphtheria virus, a pertussis virus, a pneumococcal virus, a meningococcal fusion protein, a zoster virus, a yellow fever virus, a human papillomavirus, a rabies virus, or the like.

46. The method of claim 43, wherein the eukaryotic pathogen is a fungus.

47. The method of claim 46, wherein the fungus is selected from the group consisting of Basidiomycota, Sac fungi, Eomycota, Zygomycota, Chytridiomycota, Microsporidia, Glomeromycota, Hyphomycetes, Neocallimastigomycota, Blastocladiomycota, Kickxellomycotina, Inocybe salicis, and Babjeviella inositovra.

48. The method of claim 43, wherein the eukaryotic pathogen is a protozoa.

49. The method of claim 48, wherein the protozoa is selected from the group consisting of Toxoplasma gondii, Plasmodium, Balantidium coli, Entamoeba, Entamoeba coli, Entamoeba histolytica, Entamoeba gingivalis, Entamoeba invadens, Entamoeba poleck, Giardia lamblia, Acanthamoeba, Endolimax, Endolimax nana, Iodamoeba, Iodamoeba bütschlii, Archamoebae, Entamoeba moshkovskii, Tritrichomonas, Tritrichomonas foetus, Trichomonas vaginalis, Enteromonas hominis, Entromonas, Entamoebidae, Hartmannella, Entomophthorales, Conidiobolus, Basidiobolus ranarum, Hexamitidae, Amoebida, Retortamonad, Hartminnellidae, Acanthamoebidae, Paramoeba, Conidiobolus incongruus, Conidiobolus coronatus, and Malpighamoeba.

50. The method of claim 36, wherein the pathogen is a bacterial pathogen.

51. The method of claim 50, wherein the bacterial pathogen is selected from the group consisting of Escherichia, Escherichia coli, MRSA, Shigella, Salmonella, Pseudomonas, Pseudomonas aeruginosa, Streptococcus, Streptococcus pneumoniae, Asiatic cholera, Listeria monocytogenes, Mycobacterium tuberculosis, Yersinia, Campylobacter, Klebsiella pneumoniae, Entrococcus, Neisseria, Enterobacteriaceae, Group A streptococcus, Legionella pneumophila, Corynebacterium, Staphylococcus aureus, Mycobacterium, Shigella, Helicobacter, O157:H7, Anthrax bacterium, Clostridium botulinum, Salmonella enterica, Yersinia pestis, Clostridium perfringens, Haemophilus influenzae, Rickettsia, Acinetobacter, Campylobacter jejuni, Burkholderia, Haemophilus, Enterococus faecalis, Chlamydia, Bordatella pertussis, Clostridioides difficile, Enterococcus faecium, Klebs-Löffler bacillus, Clostridium tetani, Treponema, Acinetobacter baumanni, Brucella, Treponemia pallidum, and Xanthomonas.

52. The method of any one of claims 24-51, wherein the subject experiences a reduction or elimination in an inflammatory response, a cytokine storm, or at least one off-target effect, or any combination thereof as compared to a control subject.

53. The method of any one of claims 24-52, wherein the subject experiences a reduction in a symptom or a side effect as compared to a control subject.

54. The method of claim 53, wherein the symptom is cytokine-release syndrome (CRS), a neurologic toxicity, B-cell aplasia, tumor lysis syndrome (TLS), anaphylaxis, fever, joint/muscle aches, shortness of breath, low blood pressure, confusion, or a seizure.

55. The method of any one of claims 1-54, wherein the cognate peptide is 6 to 18 amino acid residues in length.

56. The method of any one of claims 1-55, wherein the cognate peptide is selected from the group consisting of HIV Gag 77-85 (SL9) (SLYNTVATL; SEQ ID NO: 1) or SL9 variant (SLFNTIAVL; SEQ ID NO: 2), HIV Nef 190-198 (AFHHVAREL) (SEQ ID NO: 3), HIV reverse transcriptase 476-484 (ILKEPVHGV; SEQ ID NO: 4), HIV reverse transcriptase 179-187 (VIYQMDDL; SEQ ID NO: 5), CMV pp65 (495-503) (NLVPMVATV; SEQ ID NO: 6), and EBV LMP-2 (426-434) (CLGGLLTMV; SEQ ID NO: 7), or a peptide having an amino acid sequence at least 75%, at least 80%, at least 85%, at least 90%, or at least 95% identical to the sequence of one of the aforementioned peptides.
